(19) United States
(12) Patent Application Publication
(10) Pub. No.: US 2026/0363823 A1
(43) Pub. Date: Nov. 19, 2026

(54) Force Directed Graphs for Final Setups and Intermediate Staging in Clear Tray Aligners

(71) Applicant: Solventum Intellectual Properties Company, Maplewood, MN (US)

(72) Inventors: Richard E. Raby, Lino Lakes, MN (US); Michael B. Starr, Toomevara (IE); Jonathan D. Gandrud, Woodbury, MN (US)

(21) Appl. No.: 19/139,467

(22) PCT Filed: Dec. 14, 2023
(86) PCT No.: PCT/IB2023/062697

§ 371 Date: Jun. 16, 2025

Related U.S. Application Data
(60) Provisional application No. 63/432,627, filed on Dec. 14, 2022.
(60) Provisional application No. 63/460,283, filed on Apr. 18, 2023.

(51) Int. Cl. A61C 7/00; A61C 7/08

(52) U.S. Cl. CPC A61C 7/002; A61C 7/08

(57) ABSTRACT

Disclosed herein are systems automated methods for generating one or more transformations for use orthodontic treatment. The methods may receive a digital representation of a patient's dentition, comprising one or more teeth. A force directed graph (FDG) is applied to the teeth. Representations of physical interactions (e.g., a force or a moment, etc.) may be computed between teeth. These representations of physical interactions are subsequently applied to the patient's teeth, generating transforms to place the patient's teeth into orthodontic setups poses which are suitable for oral care appliance generation. These systems and methods provide an efficient and accurate approach to generating transformations for orthodontic treatments, enhancing the precision and effectiveness of the treatment process.

### RELATED DOCUMENTS

The entire disclosure of PCT Application No. PCT/IB2022/057373 is incorporated herein by reference. The entire disclosures of each of PCT Applications with Publication Nos. WO2022123402A1, WO2021245480A1, and WO2020026117A1 are incorporated herein by reference. The entire disclosure of each of the following Provisional U.S. Patent Applications is incorporated herein by reference: 63/432,627; 63/366,492; 63/366,495; 63/352,850; 63/366,490; 63/366,494; 63/370,160; 63/366,507; 63/352,877; 63/366,514; 63/366,498; 63/366,514; and 63/264,914.

### TECHNICAL FIELD

This disclosure relates to configurations and the use of force directed graphs to improve the accuracy of automatically generated clear tray aligner (CTA) devices used in orthodontic treatments.

### SUMMARY

Some existing techniques have attempted to use machine learning to generate the CTA devices, but with mixed results. As a result, there is a need for better approaches to improve the systems that automate the production of CTAs.

The present disclosure describes systems and techniques for designing and using one or more force-based models to produce intermediate stages and final setups for CTAs, in a manner which is customized to the treatment needs of the patient. Such a model is termed a “setups prediction model”. The Force-Directed Graphs (FDG) Setups prediction techniques of this disclosure may model forces between the teeth of an arch. Each tooth may be considered as a node and may undergo the application of one or more forces. In some implementations, these forces may cause nodes to interact (i.e., the trajectory or positioning of a first node may be affected by the trajectory or positioning of another node). The nodes may be iteratively moved, with the objective of bringing those nodes (e.g., teeth) into poses which are at least approximately at equilibrium, where the nodes may be in a stable configuration relative to some predefined parameters (e.g., distance from a goal configuration). The pose of a tooth may encompass at least one of the position and/or the orientation of a tooth. In some implementations, FDG Setups may be used to generate a final setup configuration of teeth. In some implementations, FDG setups may be used to generate a series of intermediate stages (intermediate configurations) of teeth. A final setup (also referred to as final setups) is a target configuration of 3D tooth representations (such as 3D tooth meshes or 3D point clouds) of the patient's teeth at the end of treatment. An intermediate setup (also referred to as an “intermediate stage” or as “intermediate staging”) describes a configuration of teeth during one of the several stages of treatment, after the teeth leave their maloccluded poses (e.g., positions and/or orientations) and before the teeth reach their final setup poses.

In a first aspect, a first computer-implemented method for generating setups for orthodontic alignment treatment is described including the steps of receiving, by one or more computer processors, a first digital representation of a patient's teeth, determining a prediction for one or more tooth movements for a final setup using a force-based predictive model that has been designed to predict one or more tooth movements for a final setup.

The first aspect can optionally generate additional outputs. For instance, the method can produce, by the one or more processors, an output state for the final setup. The method can determine, by the one or more computer processors, a difference between the one or more predicted tooth movements and the one or more reference tooth movements. The determined difference between the one or more predicted tooth movements and the one or more reference tooth movements can be used to modify or update one or more terms within the equation or equations that the force-based model comprises.

The method can generate, by the one or more computer processors, a digital representation predicting the position and orientation of the patient's teeth based on the one or more predicted tooth movements. A prediction for the movement of a tooth may be described by a transform (e.g., such as one or more of an affine transformation matrix, a translation vector, a quaternion, or one or more Euler angles). The force-based prediction model may predict each of tooth position and/or tooth orientation information. In some non-limiting examples, the force-based prediction model may predict the orientation and position information substantially concurrently. The setups prediction model may predict a setup transform for each tooth in the arch, to place each tooth in the final setup pose. The method can generate a digital representation of the patient's teeth based on the one or more reference tooth movements.

In a second aspect, a second computer-implemented method for generating setups for orthodontic alignment treatment pertains to intermediate staging prediction. Intermediate staging of teeth from a malocclusion stage to a final stage requires determining accurate individual teeth movements in a way that teeth are not colliding with each other, the teeth move toward their final state, and the teeth follow optimal and preferably short trajectories. Because each tooth has at least six degrees-of-freedom and an average arch has approximately fourteen teeth (though tooth counts may vary), finding the optimal trajectory for the teeth from the initial stage to the final stage is a large and complex problem.

The second computer-implemented method is customized to the treatment needs of the patient (e.g., as specified by a clinician, which may include technician or healthcare professional) and is described including the steps of receiving a first digital representation of a patient's teeth (and/or the transforms which indicate the maloccluded poses of those teeth), and a representation of a final setup (e.g., transforms which define the final setup poses of the teeth), and using a generator that contains a force-based model to determine a prediction for one or more tooth movements for one or more intermediate stages. Stated another way, the force-based model may be designed to predict one or more tooth movements for one or more intermediate stages. The second aspect can also include one or more of the optional features described above in reference to the first aspect.

Described herein are techniques for the automatic prediction of setups, which may provide the advantage of improving data precision and accuracy in comparison to existing techniques, enable new clinicians to be trained in the generation of effective setups, enable customized setups to be produced (e.g., which align with the specifications of clinicians and/or which align with the indications of provided oral care arguments), and provide the technical improvement of enhanced data precision in the formulation of these setups (e.g., intermediate staging).

In some implementations, a force directed graph for setups prediction may be designed to execute conditionally on interproximal reduction (IPR) information. IPR may be applied to the teeth, to enable greater packing of teeth in a final setup. The setups model may be designed to account for IPR quantities (e.g., millimeters of offset in from either or both of the mesial and distal sides of a tooth) and/or IPR cut planes (which may be used in conjunction with mesh Boolean operations to remove material on either or both of the mesial and/or distal sides of a tooth). For example, IPR cut planes may be used to modify one or more tooth meshes for one or more patient cases which are provided to the setups prediction model. This step improves the accuracy of the setups prediction model execution by improving data precision, because material is removed from the teeth which may otherwise lead to collisions (or other physical interactions) between teeth in the final setup (and result in noise in the training data). After the fully configured setups prediction model is deployed, IPR may be applied to a trial patient case, to modify the shapes of the teeth before the case is received as input to the setups prediction model. In some instances, IPR may be applied to one or more tooth meshes of a patient case before the computation of orthodontic metrics.

3D oral care representations are described herein as such because 3-dimensional representations are currently state of the art. Nevertheless, 3D oral care representations are intended to be used in a non-limiting fashion to encompass any representations of 3-dimensions or higher orders of dimensionality (e.g., 4D, 5D, etc.), and it should be appreciated that machine learning models can be trained using the techniques disclosed herein to operate on representations of higher orders of dimensionality.

In some implementations, a final setup may be used to generate, at least in part, one or more intermediate stages. Each stage may be used in the generation of a clear tray aligner. Such aligners may incrementally move the patient's teeth from the initial or maloccluded poses to the final poses represented by the final setup.

This disclosure relates to methods for generating transformations for use in orthodontic treatment. The methods may receive a digital representation of a patient's dentition (e.g., 3D meshes of the patient's teeth), and apply a force-directed graph (FDG) to the teeth to move the teeth into orthodontic setups poses. The methods may compute representations of physical interactions on the teeth (e.g., collisions, etc.), apply those representations of physical interactions to the teeth, generate transforms for one or more of the patient's teeth, and apply the transforms to the teeth. An example of a physical interaction between teeth (or between other 3D representations of oral care data) can be modelled using a virtual spring. An interaction may connect a tooth in a first stage to that same tooth in a later stage of orthodontic treatment. An interaction may connect a tooth in a first stage to a different tooth in that first stage of orthodontic treatment. In some implementations, a force may include a collision interaction between two or more teeth (or other 3D representations of oral care data in the dentition).

The methods may further include computing translations or rotations of the teeth based on the representations of physical interactions. Each tooth may be represented by a corresponding node in the FDG, and forces may act on the nodes. The forces may be modeled using Gauss's Law, a Universal Law of Gravitation, Hooke's Law, a nuclear force, or a model that describes at least one of a force or a moment (e.g. a moment which relates to a displacement). The transform for each tooth can be a setup transformation for orthodontic aligner treatment, describing the tooth's pose after completion or during the course of treatment.

In some implementations, an interaction (e.g., a spring) can be attached to a first endpoint on a first tooth, and a second endpoint on a second tooth. An endpoint can be located proximally to at least one of a surface of a tooth crown, a surface of a tooth root, a crown centroid, a root centroid, a root center of resistance, a root center of moments, a root center of area, a tooth contact point, an interproximal surface, at least a portion of an appliance, or at least one 3D representation of oral care data.

Techniques of this disclosure may generate transforms for 3D representations of oral care data, such as one or more teeth of the patient, a digital pontic tooth, a block of teeth, a crown, a root, a bridge, an implant, a bite block, an attachment, a bracket, an appliance, a restoration, or at least a portion of cortical bone, or the like.

The digital representation includes various data such as tooth dimensions, distances between adjacent teeth, 3D representations of teeth, procedure parameters, doctor preferences, tooth positions and orientations, tooth names and classifications, tooth metrics, and mesial and distal IPR values. The physical interactions can act between teeth in the same or different stages of treatment, and may involve collisions with other oral care data representations.

The computing device includes interface hardware for receiving the orthodontic treatment representation, processing circuitry for applying models of physical interactions and generating transformations, and a memory unit for storing the treatment representation.

The methods enable efficient and accurate generation of transformations for orthodontic treatment, improving the planning and execution of such treatments. The methods may, in some implementations, be used in a nearly real-time manner, such as in a clinician's office, while the patient waits.

### BRIEF DESCRIPTION OF DRAWINGS

FIG. 1 shows a method of augmenting training data for use in training machine learning (ML) models of this disclosure.

FIG. 2 shows the input and output data from an implementation of the FDG-based techniques of this disclosure.

FIG. 3 shows the results of the FDG-based techniques of this disclosure applied to a set of nodes.

FIG. 4 shows a discretization of the continuous path that a tooth may follow, when methods of this disclosure are used to generate intermediate orthodontic stages.

FIG. 5 shows an example of graph edge attachment points, in accordance with techniques of this disclosure.

FIG. 6 shows a method of training a machine learning (ML) model to generate (or modify) a data structure that describes a force-directed graph.

FIG. 7 shows a method of using a fully trained machine learning (ML) model to generate (or modify) a data structure that describes a force-directed graph.

FIG. 8 shows a 2D cross section of the 3D tooth meshes of the maloccluded arch side-by-side with the final setup arch at the end of treatment using a force-directed graph setup model.

FIG. 9 shows a 2D cross section of the 3D tooth meshes of the maloccluded arch super-imposed with the final setup arch at the end of treatment using a force-directed graph setup model.

FIG. 10 shows a zoom of FIG. 9 that illustrates attachment points and springs connecting those attachment points.

FIG. 11 shows the directions of movement of tooth centroids through the application of force directed graphs.

FIG. 12 shows a 3D arch of tooth meshes in malocclusion 1200, and a 3D arch of tooth meshes in final setup 1202, as the teeth appear after the completion of setups prediction using force-directed graphs.

FIG. 13 shows force-directed graph where each tooth has 3 attachment points.

FIG. 14 shows a force-directed graph where all degrees of freedom for each tooth may be controlled using a single spring per pair of teeth. See the springs connecting tooth centroids.

FIG. 15 shows a top-down view of LR3 and LR4, and the force-directed graph spring connecting them.

FIG. 16 shows a side view of LR3 and LR4, and the force-directed graph spring connecting them.

### DETAILED DESCRIPTION

In orthodontics, an anterior posterior (AP) shift may involve a sagittal shift of the mandible (lower arch), moving the mandible either forward or backwards. The application of the AP Shift may improve the class relationship of the teeth. Class may describe the patient's malocclusion. Possible classes include: class 1, class 2 or class 3. Elastics may aid in the shift of the mandible. Such elastics may attach to hardware on the teeth, such as buttons. In some instances, the setups prediction model of this disclosure may directly receive an AP shift transform as an input, which may improve the data precision of the resulting model. In some instances, an AP shift transform may first be applied to the patient case data before the patient case data are received as input to the setups prediction model of this disclosure.

The predictive models of the present disclosure may, in some implementations, produce more accurate results by the incorporation of one or more of the following inputs: archform information V, interproximal reduction (IPR) information U, tooth dimension information P, tooth gap information Q, latent capsule representations of oral care meshes T, latent vector representations of oral care meshes A, procedure parameters K (which may describe a clinician's intended treatment of the patient), doctor preferences L (which may describe the typical procedure parameters chosen by a doctor), flags regarding tooth status M (such as for fixed or pinned teeth), tooth position information N, tooth orientation information O, tooth name/dental notation R, oral care metrics S (comprising at least one of oral care metrics and restoration design metrics). Such inputs may be incorporated, for example, into one or more of the equations which describe the forces between 3D representations of oral care data (e.g., such as teeth) which are provided to the force-directed graphs of this disclosure, according to the techniques described herein.

Systems of this disclosure may, in some instances, be deployed in a clinical context (such as in a dental or orthodontic office) for use by clinicians (e.g., doctors, dentists, orthodontists, nurses, hygienists, oral care technicians). Such systems which are deployed in a clinical context may enable clinicians to process oral care data (such as dental scans) in the clinic environment, or in some instances, in a “chairside” context (where the patient is present in the clinical environment). A non-limiting list of examples of techniques may include: segmentation, mesh cleanup, coordinate system prediction, CTA trimline generation, restoration design generation, appliance component generation or placement or assembly, generation of other oral care meshes, the validation of oral care meshes, setups prediction, removal of hardware from tooth meshes, hardware placement on teeth, imputation of missing values, clustering on oral care data, oral care mesh classification, setups comparison, metrics calculation, or metrics visualization. The execution of these techniques may, in some instances, enable patient data to be processed, analyzed and used in appliance generation by the clinician before the patient leaves the clinical environment (which may facilitate treatment planning because feedback may be received from the patient during the treatment planning process).

Systems of this disclosure may automate operations in digital orthodontics (e.g., setups prediction, hardware placement, setups comparison), in digital dentistry (e.g., restoration design generation) or in combinations thereof. Some techniques may apply to either or both of digital orthodontics and digital dentistry. A non-limiting list of examples is as follows: segmentation, mesh cleanup, coordinate system prediction, oral care mesh validation, imputation of oral care parameters, oral care mesh generation or modification (e.g., using autoencoders, transformers, continuous normalizing flows, or denoising diffusion models), metrics visualization, appliance component placement or appliance component generation or the like. In some instances, systems of this disclosure may enable a clinician or technician to process oral care data (such as scanned dental arches). In addition to segmentation, mesh cleanup, coordinate system prediction or validation operations, the systems of this disclosure may enable orthodontic treatment planning, which may involve setups prediction as at least one operation. Systems of this disclosure may also enable restoration design generation, where one or more restored tooth designs are generated and processed in the course of creating oral care appliances. Systems of this disclosure may enable either or both of orthodontic or dental treatment planning, or may enable automation steps in the generation of either or both of orthodontic or dental appliances. Some appliances may enable both of dental and orthodontic treatment, while other appliances may enable one or the other.

Techniques of this disclosure may require evaluation of hundreds or thousands of cohort patient cases, to ensure the correct performance of the force-direct graphs. A cohort patient case may include a set of tooth crown meshes, a set of tooth root meshes, or a data file containing attributes of the case (e.g., a JSON file). A typical example of a cohort patient case may contain up to 32 crown meshes (e.g., which may each contain tens of thousands of vertices or tens of thousands of faces), up to 32 root meshes (e.g., which may each contain tens of thousands of vertices or tens of thousands of faces), multiple gingiva mesh (e.g., which may each contain hundreds of thousands of vertices or hundreds of thousands of faces) or one or more JSON files which may each contain hundreds of thousands of values (e.g., objects, arrays, strings, real values, Boolean values or Null values).

Aspects of the present disclosure can provide a technical solution to the technical problem of predicting, using force directed graphs, orthodontic setups for use in oral care appliance generation (e.g., intermediate stages or final setups for the generation of aligner trays). Aspects of the present disclosure may need to be executed in a time-constrained manner, such as when an oral care appliance must be generated for a patient immediately after intraoral scanning (e.g., while the patient waits in the clinician's office). As such, aspects of the present disclosure are necessarily rooted in the underlying computer technology of setups transform prediction for oral care appliance generation and cannot be performed by a human, even with the aid of pen and paper. For instance, implementations of the present disclosure must be capable of: 1) storing thousands or millions of mesh elements of the patient's dentition in a manner that can be processed by a computer processor and storing the transforms associated with the teeth of the patient's dentition; 2) performing calculations on thousands or millions of mesh elements, e.g., to quantify aspects of the shape and or/structure of an individual tooth in the 3D representation of the patient's dentition; and 3) predicting, based on force directed graphs, orthodontic setups for use in oral care appliance generation, and doing so during the course of a short office visit.

This disclosure pertains to digital oral care, which encompasses the fields of digital dentistry and digital orthodontics. This disclosure generally describes methods of processing three-dimensional (3D) representations of oral care data, and/or associated transforms. It should be understood, without loss of generality, that there are various types of 3D representations. One type of 3D representation is a 3D geometry. A 3D representation may include, be, or be part of one or more of a 3D polygon mesh, a 3D point cloud (e.g., such as derived from a 3D mesh), a 3D voxelized representation (e.g., a collection of voxels—for sparse processing), or 3D representations which are described by mathematical equations. Although the term “mesh” is used frequently throughout this disclosure, the term should be understood, in some implementations, to be interchangeable with other types of 3D representations. A 3D representation may describe elements of the 3D geometry and/or 3D structure of an object.

Dental arches S1, S2, S3 and S4 all contain the exact same tooth meshes, but those tooth meshes are transformed differently, according to the following description. A first arch S1 includes a set of tooth meshes arranged (e.g., using transforms) in their positions in the mouth, where the teeth are in the mal positions and orientations. A second arch S2 includes the same set of tooth meshes from S1 arranged (e.g., using transforms) in their positions in the mouth, where the teeth are in the ground truth setup positions and orientations. A third arch S3 includes the same meshes as S1 and S2, which are arranged (e.g., using transforms) in their positions in the mouth, where the teeth are in the predicted final setup poses (e.g., as predicted by one or more of the techniques of this disclosure). S4 is a counterpart to S3, where the teeth are in the poses corresponding to one of the several intermediate stages of orthodontic treatment with clear tray aligners.

It should be understood, without loss of generality, that the techniques of this disclosure which apply to final setups are also applicable to intermediate staging in orthodontic treatment, particularly Geometric Deep Learning (GDL) Setups, Reinforcement Learning (RL) Setups, Variational Autoencoder (VAE) Setups, Capsule Setups, Multilayer Perceptron (MLP) Setups, Diffusion Setups, Pose Transfer (PT) Setups, Similarity Setups, Force Directed Graphs (FDG) Setups, Transformer Setups, Setups Comparison, or Setups Classification. The Metrics Visualization aspects of this disclosure may also be configured to visualize data from both final setups and intermediate stages. MLP Setups, VAE Setups and Capsule Setups each fall within the scope of Autoencoder Setups. Some implementations of MLP Setups may fall within the Scope of Transformer Setups. Representation Setups refers to any of MLP Setups, VAE Setups, Capsule Setups and any other setups prediction machine learning model which uses an autoencoder to create the representation for at least one tooth.

Each of the setups prediction techniques of this disclosure is applicable to the fabrication of clear tray aligners and/or indirect bonding trays. The setups predictions techniques may also be applicable to other products that involve final teeth poses, also. A pose may comprise a position (or location) and a rotation (or orientation).

A 3D mesh is a data structure which may describe the geometry or shape of an object related to oral care, including but not limited to a tooth, a hardware element, or a patient's gum tissue. A 3D mesh may include one or more mesh elements such as one or more of vertices, edges, faces and combinations thereof. In some implementations, mesh element may include voxels, such as in the context of sparse mesh processing operations. Various spatial and structural features may be computed for these mesh elements and be provided to the predictive models of this disclosure, with the predictive models of this disclosure providing the technical advantage of improving data precision in the form of the models of this disclosure outputting more accurate predictions.

A patient's dentition may include one or more 3D representations of the patient's teeth (e.g., and/or associated transforms), gums and/or other oral anatomy. An Orthodontic Metric (OM) may, in some implementations, quantify the relative positions and/or orientations of at least one 3D representation of a tooth relative to at least one other 3D representation of a tooth. A Restoration Design Metric (RDM) may, in some implementations, quantify at least one aspect of the structure and/or shape of a 3D representation of a tooth. An Orthodontic Landmark (OL) may, in some implementations, locate one or more points or other structural regions of interest on a 3D representation of a tooth. An OL may, in some implementations, be used in the generation of an orthodontic or dental appliance, such as a clear tray aligner or a dental restoration appliance. A mesh element may, in some implementations, comprise at least one constituent element of a 3D representation of oral care data. For example, in the case of a tooth that is represented by a 3D mesh, mesh elements may include at least: vertices, edges, faces and voxels. A mesh element feature may, in some implementations, quantify some aspect of a 3D representation in proximity to or in relation with one or more mesh elements, as described elsewhere in this disclosure. Orthodontic Procedure Parameters (OPP) may, in some implementations, specify at least one value which defines at least one aspect of planned orthodontic treatment for the patient (e.g., specifying desired target attributes of a final setup in final setups prediction). Orthodontic Doctor Preferences (ODP) may, in some implementations, specify at least one typical value for an OPP, which may, in some instances, be derived from past cases which have been treated by one or more oral care practitioners. Restoration Design Parameters (RDP) may, in some implementations, specify at least one value which defines at least one aspect of planned dental restoration treatment for the patient (e.g., specifying desired target attributes of a tooth which is to undergo treatment with a dental restoration appliance). Doctor Restoration Design Preferences (DRDP) may, in some implementations, specify at least one typical value for an RDP, which may, in some instances, be derived from past cases which have been treated by one or more oral care practitioners.

3D oral care representations may include, but are not limited to: 1) a set of mesh element labels which may be applied to the 3D mesh elements of teeth/gums/hardware/appliance meshes (or point clouds) in the course of mesh segmentation or mesh cleanup; 2) 3D representation(s) for one or more teeth/gums/hardware/appliances for which shapes have been modified (e.g., trimmed, distorted, or filled-in) in the course of mesh segmentation or mesh cleanup; 3) one or more coordinate systems (e.g., describing one, two, three or more coordinate axes) for a single tooth or a group of teeth (such as a full arch—as with the LDE coordinate system); 4) 3D representation(s) for one or more teeth for which shapes have been modified or otherwise made suitable for use in dental restoration; 5) 3D representation(s) for one or more dental restoration appliance components; 6) one or more transforms to be applied to one or more of: dental restoration appliance library component placement relative to one or more teeth, a tooth to be placed for an orthodontic setup (either final setup or intermediate stage), a hardware element to be placed relative to one or more teeth or the like; 7) an orthodontic setup; 8) a 3D representation of a hardware element (such as facial bracket, lingual bracket, orthodontic attachment, button, hook, bite ramp, etc.) to be placed relative to one or more teeth, etc.; 8) a 3D representation of a bonding pad for a hardware element (which may be generated for a specific tooth by outlining a perimeter on the tooth, specifying a thickness to form a shell, and then subtracting-out the tooth via a Boolean operation); 9) 3D representation of a clear tray aligner (CTA); 10) the location or shape of a CTA trimline (e.g., described as either a mesh or polyline); 11) archform that describes the contours or layout of an arch of teeth (e.g., described as a 3D polyline or as a 3D mesh or surface), which may follow the incisal edges of one or more teeth, which may follow the facial surfaces of one or more teeth, which may in some implementations correspond to the maloccluded arch and in other implementations correspond to the final setup arch (the effects of malocclusion on the shape of the archform may be diminished by smoothing or averaging of the shape of the archform), which may be described by one or more control points and/or a spline; 12) 3D representation of a fixture model (e.g., a depiction of teeth and gums for use in thermoforming a clear tray aligner, or a depiction of teeth/gums/hardware for use in thermoforming an indirect bonding tray); 13) one or more latent space vectors (or latent capsules) produced by the 3D encoder stage of a 3D autoencoder which has been trained on the reconstruction of oral care meshes (e.g., a variational autoencoder which has been trained for tooth reconstruction); 14) one or more oral care metric values (e.g., such as orthodontic metrics or restoration design generation metrics) for one or more teeth; 15) one or more landmarks (e.g., 3D points) which describe the shapes and/or geometrical attributes of one or more teeth, other dentition structures or hardware structures (e.g., to be used in orthodontic setups creation or restoration appliance component generation or placement); 16) 3D representation created by scanning (e.g., optically scanning, CT scanning or MRI scanning) a 3D printed part corresponding to one or more teeth/gums/hardware/appliances (e.g., a scanned fixture model); 17) 3D printed aligners (including optional local thickness, reinforcing rib geometry, flap positioning, or the like) 18) 3D representation of the patient's dentition that was captured chairside by a clinician or medical practitioner (e.g., in a context where the 3D representation is validated chairside, before the patient leaves the clinic, so that errors can be detected and re-scans performed as necessary); 19) dental restoration tooth design (e.g., for veneers, crowns, bridges or dental restoration appliances); 20) 3D representations of one or more teeth for use in digital oral care treatment; 21) other 3D printed parts pertaining to oral care procedures or other fields; 22) IPR cut surfaces; 23) one or more orthodontic setups transforms associated with one or more IPR cut surfaces; 24) a (digital) pontic tooth design which may fill at least a portion of the space between teeth to allow room in an orthodontic setup for an erupting tooth to later emerge from the gums; or 25) a component of a fixture model (e.g., comprising fixture model components such as interproximal webbing, block-out, bite blocks, bite ramps, interproximal reinforcement, gingival ridges, torque points, power ridges, pontic tooth or dimples, among others).

The techniques of this disclosure may be advantageously combined. For example, the Setups Comparison tool may be used to compare the output of the GDL Setups model against ground truth data, compare the output of the RL Setups model against ground truth data, compare the output of the VAE Setups model against ground truth data and compare the output of the MLP Setups model against ground truth data. With each of these setups prediction models compared against ground truth data, it may be possible to determine which model gives the best performance on a certain dataset or within a given problem domain. Furthermore, the Metrics Visualization tool can enable a global view of the final setups and intermediate stages produced by one or more of the setups prediction models, with the advantage of enabling the selection of the best setups prediction model. The Metrics Visualization tool, furthermore, enables the computation of metrics which have a global scope over a set of intermediate stages. These global metrics may, in some implementations, be consumed as inputs to the force directed graphs methods for predicting setups, or to neural network-based setups prediction methods (e.g., GDL Setups, RL Setups, VAE Setups, Capsule Setups, MLP Setups, Diffusion Setups, PT Setups, Similarity Setups, among others). The global metrics may also be provided to FDG Setups. The local metrics from this disclosure (i.e., a local metric is a metric which may be computed for one stage or setup of treatment, rather than over several stages or setups) may, in some implementations, be consumed by the neural networks herein for predicting setups, with the advantage of improving predictive results. The metrics described in this disclosure may, in some implementations, be visualized using the Metric Visualization tool.

The VAE and MAE models for mesh element labelling and mesh in-filling can be advantageously combined with the setups prediction neural networks, for the purpose of mesh cleanup ahead of or during the prediction process. In some implementations, the VAE for mesh element labelling may be used to flag mesh elements for further processing, such as metrics calculation, removal or modification. In some instances, such flagged mesh elements may be provided as inputs to a setups prediction neural network, to inform that prediction model (e.g., a neural network or a force-directed graph) about important aspects of the shape and/or structure of the mesh, with the advantage of improving the performance of the resulting setups prediction model. In some implementations, mesh in-filling may cause the geometry of a tooth to become more nearly complete, enabling the better functioning of a setups prediction model (i.e., improved correctness of prediction on account of better-formed geometry). In some instances, a neural network to classify a setup (i.e., the Setups Classifier) may aid in the functioning of a setups prediction neural network, because the setups classifier tells that setups prediction neural network when the predicted setup is acceptable for use and can be provided to a method for aligner tray generation. A Setups Classifier (e.g., GDL Setups, RL Setups, VAE Setups, Capsule Setups, MLP Setups, Diffusion Setups, PT Setups, Similarity Setups and FDG Setups, among others) may aid in the generation of final setups and also in the generation of intermediate stages. Furthermore, a Setups Classifier neural network may be combined with the Metrics Visualization tool. In other implementations, a Setups Classification neural network may be combined with the Setups Comparison tool (e.g., the Setup Comparison tool may output an indication of how a setup produced in part by the Setups Classifier compares to a setup produced by another setups prediction method). In some implementations, the VAE for mesh element labelling may identify one or more mesh elements for use in a metrics calculation. The resulting metrics outputs may be visualized by the Metrics Visualization tool.

In some examples, the Setups Classifier neural network may aid in the setups prediction technique described in U.S. Patent Application No. US20210259808A1 (which is incorporated herein by reference in its entirety) or the setups prediction technique described in PCT Application with Publication No. WO2021245480A1 (which is incorporated herein by reference in its entirety) or in PCT Application No. PCT/IB2022/057373 (which is incorporated herein by reference in its entirety). The Setups Classifier would help one or more of those techniques to know when the predicted final setup is most nearly correct. In some instances, the Setups Classifier neural network may output an indication of how far away from final setup a given setup is (i.e., a progress indicator).

In some implementations, the latent space embedding vector(s) from the reconstruction VAE can be concatenated with the inputs to the setups prediction neural network described in WO2021245480A1. The latent space vectors can also be incorporated as inputs to the other setups prediction models: GDL Setups, RL Setups, VAE Setups, Capsule Setups, MLP Setups and Diffusion Setups, among others. The advantage is to impart the reconstruction characteristics (e.g., latent vector dimensions of a tooth mesh) to that neural network, hence improving the generated setups prediction.

In some examples, the various setups prediction neural networks of this disclosure may work together to produce the setups required for orthodontic treatment. For example, the GDL Setups model may produce a final setup, and the RL Setups model may use that final setup as input to produce a series of intermediate stages setups. Alternatively, the VAE Setups model (or the MLP Setups model) may create a final setup which may be used by an RL Setups model to produce a series of intermediate stages setups. In some implementations, a setup prediction may be produced by one setups prediction neural network, and then taken as input to another setups prediction neural network for further improvements and adjustments to be made. In some implementations, such improvements may be performed in iterative fashion.

In some implementations, a setups validation model, such as the model disclosed in US Provisional Application No. U.S. 63/366,495, may be involved in this iterative setups prediction loop. First a setup may be generated (e.g., using a model trained for setups prediction, such as GDL Setups, RL Setups, VAE Setups, Capsule Setups, MLP Setups, Diffusion Setups, PT Setups, Similarity Setups and FDG Setups, among others), then the setup undergoes validation. If the setup passes validation, the setup may be outputted for use. If the setup fails validation, the setup may be sent back to one or more of the setups prediction models for corrections, improvements and/or adjustments. In some instances, the setups validation model may output an indication of what is wrong with the setup, enabling the setups generation model to make an improved version upon the next iteration. The process iterates until done.

Generally speaking, in some implementations, two or more of the following techniques of the present disclosure may be combined in the course of orthodontic and/or dental treatment: GDL Setups, Setups Classification, Reinforcement Learning (RL) Setups, Setups Comparison, Autoencoder Setups (VAE Setups or Capsule Setups), VAE Mesh Element Labeling, Masked Autoencoder (MAE) Mesh In-filling, Multi-Layer Perceptron (MLP) Setups, Metrics Visualization, Imputation of Missing Oral Care Parameters Values, Tooth Classification Using Latent Vector, FDG Setups, Pose Transfer Setups, Restoration Design Metrics Calculation, Neural Network Techniques for Dental Restoration and/or Orthodontics (e.g., 3D Oral Care Representation Generation or Modification Using Transformers), Landmark-based (LB) Setups, Diffusion Setups, Imputation of Tooth Movement Procedures, Capsule Autoencoder Segmentation, Diffusion Segmentation, Similarity Setups, Validation of Oral Care Representations (e.g., using autoencoders), Coordinate System Prediction, Restoration Design Generation or 3D Oral Care Representation Generation or Modification Using Denoising Diffusion Models.

Oral care parameters may include one or more values that specify orthodontic procedure parameters, or restoration design parameters (RDP), as described herein. Oral care parameters may define one or more intended aspects of a 3D oral care representation and may be provided to an ML model to promote that ML model to generate output which may be used in the generation of oral care appliances that are suitable for the treatment of a patient. Other types of values include doctor preferences and restoration design preferences, as described herein. Doctor preferences and restoration design preferences may define the typical treatment choices or practices of a particular clinician. Restoration design preferences are subjective to a particular clinician, and so differ from restoration design parameters. In some implementations, doctor preferences or restoration design preferences may be computed by unsupervised means, such as clustering, which may determine the typical values that a clinician uses in patient treatment. Those typical values may be stored in a datastore and recalled to be provided to an automated ML model as default values (e.g., default values which may be modified before execution of the model).

For example, one clinician may prefer one value for a restoration design parameter (RDP), while another clinician may prefer a different value for that RDP, when faced with a similar diagnosis or treatment protocol. One example of such an RDP is dental restoration style. Procedure parameters and/or doctor preferences may, in some implementations, be provided to a setups prediction model for orthodontic treatment, for the purpose of improving the customization of the resulting orthodontic appliance. Restoration design parameters and doctor restoration preferences may in some implementations be used to design tooth geometry for use in the creation of a dental restoration appliance, for the purpose of improving the customization of that appliance. In addition to oral care parameters, doctor preferences, and doctor restoration preferences, some implementations of ML prediction models of this disclosure, in orthodontic treatment, may also take as input a setup (e.g., an arrangement of teeth). In some such implementations, an ML prediction model of this disclosure may take as input a final setup (i.e., final arrangement of teeth), such as in the case of a prediction model trained to generate intermediate stages. For simplicity, these preferences are referred to as doctor restoration preferences, but it is intended to be used in a non-limiting sense. Specifically, it should be appreciated that these preferences may be specified by any treating or otherwise appropriate medical professional and are not intended to be limited to doctor preferences per se (i.e., preferences from someone in possession of a D.D.S. or M.D. or equivalent degree).

An oral care professional or clinician, such as a dentist or orthodontist, may specify information about patient treatment in the form of a patient-specific set of procedure parameters. In some instances, an oral care professional may specify a set of general preferences (aka doctor preferences) for use over a broad range of cases, to use as default values in the set of procedure parameters specification process. Oral care parameters may in some implementations be incorporated into the techniques described in this disclosure, such as one or more of GDL Setups, VAE Setups, RL Setups, Setups Comparison, Setups Classification, VAE Mesh Element Labelling, MAE Mesh In-Filling, Validation Using Autoencoders, Imputation of Missing Procedure Parameters Values, Metrics Visualization, or FDG Setups. One or more of these models may take as input one or more procedure parameters vector K and/or one or more doctor preference vectors L. In some implementations, one or more of these models may introduce to one or more equations which define the force directed graph one or more procedure parameters vector K and/or one or more doctor preferences vectors L. In some implementations, one or more of these models may introduce either or both of K and L to a mathematical calculation, such as a force calculation, for the purpose of improving that calculation and the ultimate customization of the resulting appliance to the patient.

Some implementations of a force directed graph for predicting a setup (such as GDL Setup, VAE Setup or RL Setup) may incorporate information from an oral care professional (aka doctor). This information may influence the arrangement of teeth in the final setup, bringing the positions and orientations of the teeth into conformance with a specification set by the doctor, within tolerances. In some implementations of the GDL Setup model, oral care parameters may be provided directly into the generator network as a separate input alongside the mesh data. In some implementations of GDL Setups, oral care parameters may be incorporated into the feature vector which is computed for each mesh element before the mesh elements are input to the generator for processing. Some implementations of a VAE Setup model may incorporate oral care parameters into the setups predictions. In some implementations, the procedure parameters K and/or the doctor preference information L may be concatenated with the latent space vector C. A doctor's preferences (e.g., in an orthodontic context) and/or doctor's restoration preferences may be indicated in a treatment form, or they could be based upon characteristics in treatment plans such as final setup characteristics (e.g., amount of bite correction or midline correction in planned final setups), intermediate staging characteristics (e.g., treatment duration, tooth movement protocols, or overcorrection strategies), or outcomes (e.g., number of revisions/refinements).

Orthodontic procedure parameters may specify one or more of the following (with possible values shown in { }).
Teeth To Move: {AnteriorsOnly, AnteriorsAndBicuspids, FullArch}
Tooth Movement Restrictions: for each tooth, indicate if tooth is {DoNotMove, Missing, ToBeExtracted, Primary/Erupting, Clear}
Overbite: {ShowResultingOverbiteAfterAlignment, MaintainInitialOverbite, CorrectOpenBite, CorrectDeepBite}
Overjet: {ShowResultingOverjetAfterAlignment, MaintainInitialOverjet, ImproveResultingOverjet}
Anterior/Posterior (AP) Relationship
   Maintain: {Right, Left, Both}
   Improve canine relationship only: {Right, Left, Both}
   Improve canine and/or molar relationship up to 4 mm: {Right, Left, Both}
   Correct to Class I (canine and molar): {Right, Left, Both}
Crossbite (if present)
   Anterior: {DoNotCorrect, Correct, N/A}
   Posterior: {DoNotCorrect, Correct, N/A}
Correction to Class I (canine and molar): {Right, Left, Both}
   Correct with Posterior IPR: {yes, no}
      Class II/III correction simulation (elastics required): {yes, no}
      Sequential Distalization (elastic recommended): {yes, no}
      Include cuts for elastics?: {yes, no}
      Preferred cuts for elastics: {UseButtonCutoutsOnMolarsAndHooksOnCanines, UseButtonCutoutsOnly, UseHooksOnly}
      Stage to start cuts for elastics: [integer]
LevelingOfUpperAnteriors: {Laterals0.5mmShorterThanCentral, LevelIncisalEdges, LevelGingivalMargins, AsIndicated}
Spacing: {CloseAllSpaces, LeaveSpecificSpaces}
Preferred Midline Position: {SetTheUpperMidlineToldeal, MatchTheUpperAndLowerToEachOther}
Resolve Upper Crowding by Expand: {Primarily, AsNeeded, None}
Resolve Upper Crowding by Procline: {Primarily, AsNeeded, None}
Resolve Upper Crowding by IPR—Anterior: {Primarily, AsNeeded, None}
Resolve Upper Crowding by IPR—Posterior Right: {Primarily, AsNeeded, None}
Resolve Upper Crowding by IPR—Posterior Left: {Primarily, AsNeeded, None}
Resolve Lower Crowding by Expand: {Primarily, AsNeeded, None}
Resolve Lower Crowding by Procline: {Primarily, AsNeeded, None}
Resolve Lower Crowding by IPR—Anterior: {Primarily, AsNeeded, None}
Resolve Lower Crowding by IPR—Posterior Right: {Primarily, AsNeeded, None}
Resolve Lower Crowding by IPR—Posterior Left: {Primarily, AsNeeded, None}
Finishing Arch Form: {Patient'sNatural, AsIndicated}
   [doctor can specify an archform—selected from a set of options or custom-designed]

Other orthodontic procedure parameters may be defined, such as those which may be used to place standardized brackets at prescribed occlusal heights on the teeth. In some implementations, one or more orthodontic procedure parameters may be defined to specify at least one of the 2^{nd} and 3^{rd} order rotation angles to be applied to a tooth (i.e., angulation and torque, respectively), which may enable a target setup arrangement where crown landmarks lie within a threshold distance of a common occlusal plane, for example. In some implementations, one or more orthodontic procedure parameters may be defined to specify the position in global coordinates where at least one landmark (e.g., a centroid) of a tooth crown (or root) is to be placed in a setup arrangement of teeth. Generally, an oral care parameter may be defined which corresponds to an oral care metric. For example, an orthodontic procedure parameter may be defined which corresponds to an orthodontic metric (e.g., to specify at the input of a setups prediction model an amount of a certain metric which is desired to appear in a predicted setup).

Doctor preferences may differ from orthodontic procedure parameters in that doctor preferences pertain to an oral care provider and may comprise of the means, modes, medians, minimums, or maximums (or some other statistic) of past settings associated with an oral care provider's treatment decisions on past orthodontic cases. Procedure parameters, on the other hand, may pertain to a specific patient, and describe the needs of a particular patient's treatment. Doctor preferences may pertain to a doctor and the doctor's past treatment practices, whereas procedure parameters may pertain to the treatment of a particular patient. Doctor preferences (or “treatment preferences”) may specify one or more of the following (with some non-limiting possible values shown in { }). Other possible values are found elsewhere in this disclosure.

Doctor preferences may specify one or more of the following (with other possible values found elsewhere in this disclosure).
Deep Bite Cases (Amount of Bite Correction)—Final Overbite: [real value in millimeters, e.g., 0.5 mm]
   Option—Intrude Upper Anteriors: {yes, no}
   Option—Include lower canines in vertical overcorrection: {yes, no}
Midline Correction in Planned Final Setup: {MaintainInitialMidline, ImproveMidlineWithIPR, AsIndicated}
Deep Bite Cases—Reverse Curve of Speed: {yes, no}
Anterior Open Bite Cases—Final Overbite: [real value in millimeters, e.g., 2 mm]
   Is Arch Expansion a Priority for Your Cases?: {Yes, No}
   If yes, specify acceptable expansion per quadrant in mm.
When expanding upper molars, apply buccal root torque: {yes, no}
Is IPR Acceptable of First Tx Design: {yes, no}
Maximum IPR per contact:
   Upper Anterior: [specify in mm]
   Lower Anterior: [specify in mm]
   Upper and Lower Anterior: [specify in mm]
Is Asymmetric IPR Acceptable?: {yes, no}
Final Tooth Position (Overcorrection Strategy): {Ideal, Overcorrected}
Root Movement: {MoveRootsAsNeededToAchieveTreatmentGoals, LimitPosteriorRootMovement, LimitAllRootMovement}
Final Occlusal Contacts: {AllContactsBalancedWhenPossible, NoOcclusalContactOnUpperIncisors, FinishWithHeavyPosteriorContacts, Other}
Is Asymmetric AP Shift Acceptable for Class Correction?: {yes, no, other}
Treatment Duration: [count of stages]
Tooth Movement Protocol: {protocol_A, protocol_B, protocol_C}

Existing techniques have attempted to move the teeth towards an archform V after a setup prediction has already been rendered by other method components, which introduces error to the resulting setup and diminishes performance in view of the purpose of the method components which made the setups prediction. The present disclosure provides several improvements over these existing techniques by enabling archform information to be introduced directly into the setups prediction neural network as an input to that neural network, with the technical improvement of providing setups predictions that more accurately meets the orthodontic treatment needs of the patient (thereby improving data precision). Archform information V may be provided as an input to any of the GDL Setups, RL Setups, VAE Setups, Capsule Setups, MLP Setups and Diffusion Setups prediction neural networks. In some implementations, archform information V may be provided directly to one or more internal neural network layers in one or more of those setups applications.

The additional procedure parameters may include text descriptions of the patient's medical condition and of the intended treatment. Such text descriptions may be analyzed via natural language processing operations, including tokenization, stop word removal, stemming, n-gram formation, text data vectorization, bag of words analysis, term frequency inverse document frequency (TF-IDF) analysis, sentiment analysis, naive Bayes classification, and/or logistic regression classification. The outputs of such analysis techniques may be used as input to one or more of the neural networks of this disclosure with the advantage of customizing and improving the predicted outputs (e.g., the predicted setups or predicted mesh geometries).

These additional orthodontic parameters and doctor preferences may also be incorporated into the neural networks of this disclosure, with the data precision and efficiency-related advantages of improving the predictive power of those neural networks and enabling those neural networks to predict output examples which better match the treatment needs of the individual patient.

In some implementations, a dataset used for training one or more of the neural network models of this disclosure may be filtered conditionally on one or more of the orthodontic procedure parameters described in this section. In some instances, patient cases which exhibit outlier values for one or more of these procedure parameters may be omitted from a dataset (alternatively used to form a dataset) for training one or more of the neural networks of this disclosure.

One or more procedure parameters and/or doctor preferences may be provided to a neural network during training. In this manner the neural network may be conditioned on the one or more procedure parameters and/or doctor preferences. Examples of such neural networks include a conditional generative adversarial network (cGAN) and/or a conditional variational autoencoder (cVAE), either of which may be used for the various neural network-based applications of this disclosure.

In some instances, tooth shape-based inputs may be provided to an equation for a force directed graph for setups predictions. In other instances, non-shape-based inputs can be used, such as a tooth name or designation, as it pertains to dental notation. In some implementations, a vector R of flags may be provided to an equation for a force directed graph, where a ‘1’ value indicates that the tooth is present and a ‘0’ value indicates that the tooth is absent from the patient case (though other values are possible). The vector R may comprise a 1-hot vector, where each element in the vector corresponds to a tooth type, name or designation. Identifying information about a tooth (e.g., the tooth's name) can be provided to the predictive models of this disclosure, with the advantage of enabling models to configured to handle different teeth in tooth-specific ways. For example, the setups prediction model may be configured to make setups transformations predictions for a specific tooth designation (e.g., upper right central incisor, or lower left cuspid, etc.). In the case of mesh cleanup autoencoders (either for labelling mesh element or for in-filling missing mesh data), the autoencoder may be trained to provide specialized treatment to a tooth according to that tooth's designation, in this manner. In the case of a setups classification neural network, a listing of tooth name(s) present in the patient's arch may better enable the neural network to output an accurate determination of setup classification, because tooth designation is a valuable input to training such a neural network. Tooth designation/name may be defined, for example, according to the Universal Numbering System, Palmer System, or the FDI World Dental Federation notation (ISO 3950).

In one example, where all except the (up to four) wisdom teeth are present in the case, a vector R may be defined as an optional input to the setups prediction neural networks of this disclosure, where there is a 0 in the vector element corresponding to each of the wisdom teeth UR8, UL8, LL8, LR8, and a 1 in the elements corresponding to the following other teeth: UR7, UR6, UR5, UR4, UR3, UR2, UR1, UL1, UL2, UL3, UL4, UL5, UL6, UL7, LL7, LL6, LL5, LL4, LL3, LL2, LL1, LR1, LR2, LR3, LR4, LR5, LR6, LR7

In some instances, the position of the tooth tip may be provided to a predictive model for setups predictions. In other instances, one or more vectors S of the orthodontic metrics described elsewhere in this disclosure may be provided to a predictive model for setups predictions. The advantage is an improved capacity for the predictive model to be configured to understand the state of a maloccluded setup and therefore be able to predict a more accurate final setup or intermediate stage.

In some implementations, the predictive model may take as input one or more indications of interproximal reduction (IPR) U, which may indicate the amount of enamel that is to be removed from a tooth during the course of orthodontic treatment (either mesially or distally). In some implementations, IPR information (e.g., amount of IPR that is to be performed on one or more teeth, as measured in millimeters, or one or more binary flags to indicate whether or not IPR is to be performed on each tooth identified by flagging) may be provided to an equation that describes a force directed graph (e.g., for setups prediction). Alternatively, the IPR information may be concatenated with a latent vector A which is produced by a VAE or a latent capsule T autoencoder. The vector(s) and/or capsule(s) resulting from such a concatenation may be provided to one or more of the predictive models of the present disclosure, with the technical improvement or added advantage of enabling the predictive models to account for IPR. IPR is especially relevant to setups prediction methods, which may determine the positions and poses of teeth at the end of treatment or during one or more stages during treatment. It is important to account for the amount of enamel that is to be removed ahead of predicted tooth movements.

In some implementations, one or more procedure parameters K and/or doctor preferences vectors L may be provided to a setups prediction model (e.g., an equation for a force directed graph). In some implementations, one or more optional vectors or values of tooth position N (e.g., XYZ coordinates, in either tooth local or global coordinates), tooth orientation O (e.g., pose, such as in transformation matrices or quaternions, Euler angles or other forms described herein), dimensions of teeth P (e.g., length, width, height, circumference, diameter, diagonal measure, volume—any of which dimensions may be normalized in comparison to another tooth or teeth), distance between adjacent teeth Q, may be provided to the predictive models of this disclosure. The “dimensions of teeth P” may in some instances be used to describe the intended dimensions of a tooth for dental restoration design generation.

In some implementations, tooth dimensions P (e.g., length, width, height, or circumference) may be measured inside a plane, such as the plane that intersects the centroid of the tooth, or the plane that intersects a center point that is located midway between the centroid and either the incisal-most extent or the gingival-most extent of the tooth. The tooth dimension of height may be measured as the distance from gums to incisal edge. The tooth dimension of width may be measured as the distance from the mesial extent to the distal extent of the tooth. In some implementations, the circularity or roundness of the tooth cross-section may be measured and included in the vector P. Circularity or roundness may be defined as the ratio of the radii of inscribed and circumscribed circles.

The distance Q between adjacent teeth can be implemented in different ways (and computed using different distance definitions, such as Euclidean or geodesic). In some implementations, a distance Q1 may be measured as an averaged distance between the mesh elements of two adjacent teeth. In some implementations, a distance Q2 may be measured as the distance between the centers or centroids of two adjacent teeth. In some implementations, a distance Q3 may be measured between the mesh elements of closest approach between two adjacent teeth. In some implementations, a distance Q4 may be measured between the cusp tips of two adjacent teeth. Teeth may, in some implementations, be considered adjacent within an arch. Teeth may, in some implementations, also be considered adjacent between opposing arches. In some implementations, any of Q1, Q2, Q3 and Q4 may be divided by a term for the purpose of normalizing the resulting value of Q. In some implementations, the normalizing term may involve one or more of: the volume of a tooth, the count of mesh elements in a tooth, the surface area of a tooth, the cross-sectional area of a tooth (e.g., as projected into the XY plane), or some other term related to tooth size.

Other information about the patient's dentition or treatment needs (or related parameters) may be concatenated with the other input vectors to one or more of an equation for a force-directed graph, MLP, GAN, generator, encoder structure, decoder structure, transformer, VAE, conditional VAE, regularized VAE, 3D U-Net, capsule autoencoder, diffusion model, and/or any of the predictive models disclosed herein.

The vector M may contain flags which apply to one or more teeth. In some implementations, M contains at least one flag for each tooth to indicate whether the tooth is pinned. In some implementations, M contains at least one flag for each tooth to indicate whether the tooth is fixed. In some implementations, M contains at least one flag for each tooth to indicate whether the tooth is a pontic. Other and additional flags are possible for teeth, as are combinations of fixed, pinned and pontic flags. A flag that is set to a value that indicates that a tooth should be fixed is a signal to the network that the tooth should not move over the course of treatment. In some implementations, an equation for a force-directed graph may be configured to be penalized for any movement in the indicated teeth (and in some particular cases, may be heavily penalized). A flag to indicate that a tooth is pontic informs the predictive model that the tooth gap is to be maintained, although that gap is allowed to move. In some cases, M may contain a flag indicating that a tooth is missing. In some implementations, the presence of one or more fixed teeth in an arch may aid in setups prediction, because the one or more fixed teeth may provide an anchor for the poses of the other teeth in the arch (i.e., may provide a fixed reference for the pose transformations of one or more of the other teeth in the arch). In some implementations, one or more teeth may be intentionally fixed, so as to provide an anchor against which the other teeth may be positioned. In some implementations, a 3D representation (such as a mesh) which corresponds to the gums may be introduced, to provide a reference point against which teeth can be moved.

Without the loss of generality, one or more of the optional input vectors K, L, M, N, O, P, Q, R, S, U and V described elsewhere in this disclosure may also be provided to the input of the predictive models of this disclosure. In particular, these optional vectors may be provided to the force-directed graphs, MLP Setups, GDL Setups, RL Setups, VAE Setups, Capsule Setups and/or Diffusion Setups, with the advantage of enabling the respective model to generate setups which better meet the orthodontic treatment needs of the patient. In the neural network-based setups implementations, such inputs may be provided, for example, by being concatenated with one or more latent vectors A which are also provided to one or more of the predictive models of this disclosure. In some implementations, such inputs may be introduced, for example, by being concatenated with one or more latent capsules T which are also provided to one or more of the predictive models of this disclosure. In the neural network-based setups implementations, one or more of K, L, M, N, O, P, Q, R, S, U and V may be provided to the neural network (e.g., MLP or Transformer) directly in a hidden layer of the network. In some instances, one or more of K, L, M, N, O, P, Q, R, S, U and V may be provided directly into the internal processing of an encoder structure. In the force-direct graphs-based implementations of setups prediction models, the inputs may be introduced into one or more terms of an equation.

In some implementations, a setups prediction model (such as GDL Setups, RL Setups, VAE Setups, Capsule Setups, MLP Setups, PT Setups, Similarity Setups and Diffusion Setups) may take as input one or more latent vectors A which correspond to one or more input oral care meshes (e.g., such as tooth meshes). In some implementations, a setups prediction model (such as GDL Setups, RL Setups, VAE Setups, Capsule Setups, MLP Setups and Diffusion Setups) may take as input one or more latent capsules T which correspond to one or more input oral care meshes (e.g., such as tooth meshes). In some implementations, a setups prediction method may take as input both of A and T.

Some implementations of the setups prediction models (e.g., GDL Setups, RL Setups, VAE Setups, Capsule Setups, MLP Setups, Diffusion Setups, PT Setups, Similarity Setups, or FDG Setup, or other setups prediction network architectures) of this disclosure may take additional inputs to aid in setups prediction. Some of these inputs may reflect the geometrical attributes of one or more teeth or of a whole arch. In some implementations, an archform or arch curve may be provided to a setups prediction model, with the technical improvement of aiding that setups prediction model in generating a suitable set of final setups poses for the teeth in a patient case (with the technical improvements being directed to both resource footprint reduction by way of more efficient location capabilities and/or data precision in the form of locating a more pertinent final setup). The archform or arch curve may be encoded as a spline, a B-spline, NonUniform Rational B-Splines (NURBS), polynomial spline, non-polynomial spline, parabolic curve, hyperbolic curve, other parameterized curve, or a piecewise linear interpolation of a parameterized curve, such as a polyline or a contiguous series of points representing a connected series of line segments. Such a curve may be computed as an average of multiple exemplars, such as exemplary final setups. Another non-limiting example of an archform is a Beta curve. In the case of force-directed graphs for setups prediction, archform information may be introduced to one or more of the equations which define the force-directed graph. In the case of the Setups VAE, the arch information may be provided to the encoder E2, as an additional input alongside E and D. In the case of the GDL Setups neural network, the arch information may be provided to the generator as an additional input to the mesh element lists and associated mesh element feature vectors. In some implementations, an archform may be described by one or more 3D representations, such as a 3D mesh, a set of 3D control points and/or as a 3D polyline. For example, the vertices of a polyline may describe an archform. In some implementations, a spring force may be modeled between a tooth and that archform, to influence the path taken by the tooth over the course of a series of intermediate stages. In some implementations, a Frenet frame may be overlaid onto an archform. The Frenet frame may locally describe the coordinate system corresponding to each point along the archform. Such a coordinate system may, in some implementations, be right-handed (or alternatively, in other implementations, left-handed). Such a coordinate system may, in some implementations, be determined, at least in part, by at least one of the tangent to the archform at the point and the archform's curvature. In some implementations, a point may be described using an LDE coordinate frame relative to an archform, where L, D and E correspond to 1) Length along the curve of the archform, 2) Distance away from the archform, and 3) Distance in the direction perpendicular to the L and D axes (which may be termed Eminence), respectively. Other geometrical inputs may also aid in the training of a setups prediction models.

As described herein, tooth movements may specify one or more tooth transformations that can be encoded in various ways to specify tooth positions and orientations within the setup and are applied to 3D representations of teeth. For instance, according to particular implementations, the tooth positions can be Cartesian coordinates of a tooth's canonical origin location which is defined in some semantic context. Tooth orientations can be represented as rotation matrices, unit quaternions, or other 3D rotation representations such as Euler angles with respect to a frame of reference (either global or local). Dimensions are real valued 3D spatial extents and gaps can be binary presence indicators or real valued gap sizes between teeth especially in instances when certain teeth are missing. In some implementations, tooth rotations may be described by 3×3 matrices (or by matrices of other dimensions). Tooth position and rotation information may, in some implementations, be combined into the same transform matrix, for example, as a 4×4 matrix, which may reflect homogenous coordinates. In some instances, affine spatial transformation matrices may be used to describe tooth transformations, for example, the transformations which describe the maloccluded pose of a tooth, an intermediate pose of a tooth and/or a final setup pose of a tooth. Some implementations may use relative coordinates, where setup transformations are predicted relative to malocclusion coordinate systems (e.g., a malocclusion-to-setup transformation is predicted instead of a setup coordinate system directly). Other implementations may use absolute coordinates, where setup coordinate systems are predicted directly for each tooth. In the relative mode, transforms can be computed with respect to the centroid of each tooth mesh (vs the global origin), which is termed “relative local.” Some of the advantages of using relative local coordinates include eliminating the need for malocclusion coordinate systems (landmarking data) which may not be available for all patient case datasets. Some of the advantages of using absolute coordinates include simplifying the data preprocessing as mesh data are originally represented as relative to the global origin. These details about tooth position encoding and tooth orientation encoding may, in some implementations, also apply one or more of the neural networks models of the present disclosure, including but not limited to: GDL Setups, RL Setups, VAE Setups, Capsule Setups, MLP Setups, Diffusion Setups, PT Setups, Similarity Setups, FDG Setups, Setups Classification, Setups Comparison, VAE Mesh Element Labeling, MAE Mesh In-filling, Mesh Reconstruction VAE, and Validation Using Autoencoders.

Representation generation neural networks based on autoencoders, U-Nets, transformers, other types of encoder-decoder structures, convolution and/or pooling layers, or other models may benefit from the use of oral care arguments (e.g., oral care metrics or oral care parameters). Force-direct graphs models may also benefit from the use of oral care arguments. For example, oral care metrics (e.g., orthodontic metrics or restoration design metrics) may convey aspects of the shape and/or structure of the patient's dentition (e.g., the shape and/or structure of an individual tooth, or the special relationships between two or more teeth) to the predictive models of this disclosure. Each oral care metric describes distinct information about the patient's dentition that may not be redundantly present in other input data that are provided to the predictive models. For example, an “Overbite” metric may quantify the overlap between the upper and lower central incisors along the vertical Z-axis, converting that information into a form which may be readily provided to the equations of a force-directed graph. In neural networks-based methods, this information may not otherwise, in some implementations, be readily ascertainable by a traditional neural network. Stated another way, the oral care metrics provide refined information about the patient's dentition that a traditional predictive model (e.g., a representation generation neural network) may not be adequately trained or configured to extract the oral care metrics described herein. However, a neural network which is specifically trained to generate oral care metrics may overcome such a shortcoming, because, for example loss may be computed in such a way as to facilitate accurate oral care metrics prediction. Oral care metrics may provide a processed version of the structure and/or shape of the patient's dentition, data which may not otherwise be available to the predictive models of this disclosure (e.g., the force-directed graphs models). In neural networks methods, this processed information is often more accessible, or more amenable for the neural network to encode into the weights of the neural network. Furthermore, in force-directed graphs methods, the processed information is often more accessible, or more amenable to influence the execution of the equations of the force-directed graph. A system disclosing the techniques disclosed herein has been utilized to run a number of experiments on 3D representations of teeth. For example, oral care metrics have been provided to a representation generation neural network which is based on a U-Net model. Based on experiments, it was found that systems using oral care metrics (e.g., “Overbite”, “Overjet” and “Canine Class Relationship” metrics) were at least 2.5% more accurate than systems that did not. Furthermore, training converges more quickly when the oral care metrics are used. Stated another way, the machine learning models trained using oral care metrics tended to be more accurate more quickly (at earlier epochs) than systems which did not. For a system which has previously been 91% accurate, an improvement in accuracy of 2.5% reduces the actual error rate by almost 30%.

PCT Application with Publication No. WO2020026117A1 is incorporated herein by reference in its entirety. WO2020026117A1 lists some examples of Orthodontic Metrics (OM). Further examples are disclosed herein. The orthodontic metrics may be used to quantify the physical arrangement of an arch of teeth for the purpose of orthodontic treatment (as opposed to restoration design metrics—which pertain to dentistry and describe the shape and/or form of one or more pre-restoration teeth, for the purpose of supporting dental restoration). These orthodontic metrics can measure how badly maloccluded the arch is, or conversely the metrics can measure how correctly arranged the teeth are. In some implementations, a force-directed graphs setup model (or GDL Setups, RL Setups, VAE Setups, Capsule Setups, MLP Setups, Diffusion Setups, PT Setups, or Similarity Setups) may incorporate one or more of these orthodontic metrics, or other similar or related orthodontic metrics. In some implementations of setups prediction models, the orthodontic metrics may be incorporated into terms of the equations that describe a force-directed setups model. The oral care metrics may also be provided to other types of implementations in digital oral care, such as neural network-based setups prediction methods. In some applications (e.g., with neural networks), such orthodontic metrics may be incorporated into the feature vector for a mesh element, where these per-element feature vectors are provided to the setups prediction network as inputs. In some neural networks implementations, such orthodontic metrics may be directly consumed by a generator, an MLP, a transformer, or other neural network as direct inputs (such as presented in one or more input vectors of real numbers S, such as described elsewhere in this disclosure. The use of such orthodontic metrics in the configuring of a predictive model may improve the performance (i.e., correctness) of the resulting predictive model, resulting in predicted transforms which place teeth more nearly in the correct final setups poses than would otherwise be possible. Such orthodontic metrics may be consumed by an encoder structure or by a U-Net structure (in the case of GDL Setups). In neural networks applications, such orthodontic metrics may be consumed by an autoencoder, variational autoencoder, masked autoencoder or regularized autoencoder (in the case of the VAE Setups, VAE Mesh Element Labelling, MAE Mesh In-Filling). Such orthodontic metrics may be consumed by a neural network which generates action predictions as a part of a reinforcement learning RL Setups model. Such orthodontic metrics may be provided to a classifier which applies a label to a setup arch (e.g., labels such as mal, staging or final setup). This description is non-limiting, as the orthodontic metrics may also be incorporated in other ways into the various techniques of this disclosure.

In neural networks application in digital oral care, the various loss calculations of the present disclosure may, in some examples, incorporate one or more orthodontic metrics, with the advantage of improving the correctness of the resulting neural network. An orthodontic metric may be used to directly compare a predicted example to the corresponding ground truth example (such as is done with the metrics in the Setups Comparison description). In other examples, one or more orthodontic metrics may be taken from this section and incorporated into a loss computation. Such an orthodontic metric may be computed on the predicted example, and then the orthodontic metric would also be computed on the ground truth example. These two orthodontic metrics results would then be provided to the loss computation, with the advantage of improving the performance of the resulting neural network. In some implementations, one or more orthodontic metrics pertaining to the alignment of two or more adjacent teeth may be computed and incorporated into a loss function, for example, to train, at least in part, a setups prediction neural network. In some implementations, such an orthodontic metric may promote the network in aligning the mesial surface of a tooth with the distal surface of an adjacent tooth. Backpropagation is an example algorithm by which a neural network may be trained using one or more loss values.

In some implementations, one or more orthodontic metrics may be used to evaluate the predicted output of a predictive model (e.g., a force-directed graphs-based setups prediction model). Such a metric(s) may enable the evaluation algorithm to determine how close the predicted output is to an acceptable output, for example, in a quantified sense. In some implementations, this use of an orthodontic metric may enable a loss value to be computed which does not depend entirely on a comparison to a ground truth. In some implementations, such a use of an orthodontic metric may enable loss calculation and network training to proceed without the need for a comparison against a ground truth example. The advantage of such an approach is that loss may be computed based on a general principle or specification for the predicted output (such as a setup) rather than tying loss calculation to a specific ground truth example (which may have been defined by a particular doctor, clinician, or technician, whose treatment philosophy may differ from that of other technicians or doctors). In some implementations, such an orthodontic metric may be defined based on a FID (Frechet Inception Distance) score.

The following is a description of some of the orthodontic metrics which are used to quantify the state of a set of teeth in an arch for the purpose of orthodontic treatment. These orthodontic metrics indicate the degree of malocclusion that the teeth are in at a given stage of clear tray aligner treatment.

An orthodontic metric that can be computed using tensors may be especially advantageous when configuring (or provisioning) the force-directed graphs of the present disclosure, because tensor operations may promote efficient computations. The more efficient (and faster) the computation, the faster the rate at which training can proceed.

In some examples, an error pattern may be identified in one or more predicted outputs of a predictive model (e.g., a transformation matrix for a predicted tooth setup, a labelling of mesh elements for mesh cleanup, an addition of mesh elements to a mesh for the purpose of mesh in-filling, a classification label for a setup, a classification label for a tooth mesh, etc.). One or more orthodontic metrics may be selected to become an input to the next round of predictive model execution, to address any pattern of errors or deficiencies which may be identified in the one or more predicted outputs.

Some OM may be defined relative to an arch form coordinate frame, the LDE coordinate system. In some implementations, a point may be described using an LDE coordinate frame relative to an archform, where L, D and E correspond to 1) Length along the curve of the archform, 2) Distance away from the archform, and 3) distance in the direction perpendicular to the L and D axes (which may be termed Eminence), respectively.

Variations of the OM and other techniques of the present disclosure may compute collisions between 3D representations (e.g., of oral care objects, such as teeth). Such collisions may be computed as at least one of: 1) penetration distance between 3D tooth representations, 2) count of overlapping mesh elements between 3D tooth representations, and 3) volume of overlap between 3D tooth representations. In some implementations, an OM may be defined to quantify the collision of two or more 3D representations of oral care structures, such as teeth. Some optimization algorithms, such as setups prediction techniques, may seek to minimize collisions between oral care structures (such as teeth). Between-arch orthodontic metrics are as follows.

Six (6) metrics for the comparison of two or more arches are listed below. Other suitable comparison orthodontic metrics are found elsewhere in this disclosure, such as in the section for the Setups Comparison technique.
1. Rotation geodesic distance (rotation between predicted example and ground truth setup example)
2. Translation distance (gap between predicted example and ground truth setup example)
3. Normalized translation distance
4. 3D alignment error that measures the distance between predicted mesh elements and ground truth mesh elements, in units of mm.
5. Normalized 3D alignment
6. Percent overlap (% overlap) by volume (alternatively % overlap by mesh elements) of predicted example and corresponding ground truth example

Within-arch orthodontic metrics are as follows.

Alignment—A 3D tooth orientation vector may be calculated using the tooth's mesial-distal axis. A 3D vector, which may be tangent to the archform at the position of the tooth may also be calculated. The XY components (i.e., which may be 2D vectors) may then be used to compare the orientation of the archform at the tooth's location to the tooth's orientation in XY space. Cosine similarity may be used to calculate the 2D orientation difference (angle) between the archform tangent and the tooth's mesial-distal axis.

Arch Symmetry—For each left-right pair of teeth (e.g., lower left lateral incisor and/or lower right lateral incisor) the absolute difference may be calculated between each tooth's X-coordinate and the global coordinate reference frame's X-axis. This delta may indicate the arch asymmetry for a given tooth pair. The result of such a calculation may be the mean X-axis delta of one or more tooth-pairs from the arch. This calculation may, in some implementations, be performed relative to the Y-axis with y-coordinates (and/or relative to the Z axis with Z-coordinates).

Archform D-axis Differences—May compute the D dimension difference (i.e., the positional difference in the facial-lingual direction) between two arch states, for one or more teeth. May, in some implementations, return a dictionary of the D-direction tooth movement for each tooth, with tooth UNS number as the key. May use the LDE coordinate system relative to an archform.

Archform (Lower) Length Ratio—May compute the ratio between the current lower arch length and the arch length as it was in the original maloccluded lower arch.

Archform (Upper) Length Ratio—May compute the ratio between the current upper arch length and the arch length as it was in the original maloccluded upper arch.

Archform Parallelism (Full arch)—For at least one local tooth coordinate system origin in the upper arch, the one or more nearest origins (e.g., tooth local coordinate system origins) in the lower arch. In some implementations, the two nearest origins may be used. May compute the straight line distance from the upper arch point to the line formed between the origins of the two teeth in the opposing (lower) arch. May return the standard deviation of the set of “point-to-line” distances mentioned above, where the set may be composed of the point-to-line distances for each tooth in the arch.

Archform Parallelism (Individual tooth)—This metric may share some computational elements with the archform_parallelism_global orthodontic metric, except that this metric may input the mean distance from a tooth origin to the line formed by the neighboring teeth in opposing arches (e.g., a tooth in the upper arch and the corresponding tooth in the lower arch). The mean distance may be computed for one or more such pairs of teeth. In some implementations, this may be computed for all pairs of teeth. Then the mean distance may be subtracted from the distance that is computed for each tooth pair. This OM may yield the deviation of a tooth from a “typical” tooth parallelism in the arch.

Buccolingual Inclination—For at least one molar or premolar, find the corresponding tooth on the opposite side of the same arch (i.e., for a tooth on the left side of the arch, find the same type of tooth on the right side and vice versa). This OM may compute an n-element list for each tooth (e.g. n may equal 2). This list may contain at least the tooth IDs of the teeth in each pair of teeth (e.g., LeftLowerFirstMolar and RightLowerFirstMolar in a list=[left_tooth_idx_1, right_tooth_idx_2]). Such an n-element vector may be computed for each molar and each premolar in the upper and lower arches. The buccal cusps may be identified on the molars and premolars on each of the left and right sides of the arch. Draw a line between the buccal cusps of the left tooth and the buccal cusps on the right tooth. Make a plane using this line and the z-axis of the arch. The lingual cusps may be projected onto the plane (i.e., at this point the angle of inclination may be determined). By performing an additional projection, the approximate vertical distance between the lingual cusps and the buccal cusps may be computed. This distance may be used as the buccolingual inclination OM.

Canine Overbite—The upper and lower canines may be identified. The first premolar for the given side of the mouth may be identified. On a given side of the arch, a distance may be computed between the upper canine and the lower canine, and also between the upper pre-molar and the lower pre-molar. The average (or median, or mode or some other statistic) may be computed for the measured distances. The z-component of this result indicates the degree of overbite. Overbite may be computed between any tooth in one arch and the corresponding tooth in the other arch.

Canine Overjet Contact—May calculate the collisions (e.g., collision distances) between pairs of canines on opposing arches.

Canine Overjet Contact KDE—May take an orthodontic metric score for the current patient case as input and may convert that score into to a log-likelihood using a previously trained kernel density estimation (KDE) model or distribution. This operation may yield information about where in the distribution of “typical” values this patient case lies.

Canine Overjet—This OM may share some computational steps with the canine overbite OM. In some implementations, average distances may be computed. In some implementations, the distance calculation may compute the Euclidean distance of the XY components of a tooth in the upper arch and a tooth in the lower arch, to yield overjet (i.e., as opposed to computing the difference in Z-components, as may be performed for canine overbite). Overjet may be computed between any tooth in one arch and the corresponding tooth in the other arch.

Canine Class Relationship (also applies to first, second and third molars)—This OM may, in some implementations comprise two functions (e.g., written in Python).
get_canine_landmarks( ): Get landmarks for each tooth which may be used to compute the class relationship, and then, in some implementations, map those landmarks onto the global coordinate space so that measurements may be made between teeth.
class_relationship_score_by_side( ): May compute the average position of at least one landmark on at least one tooth in the lower arch, and may compute the same for the upper arch. Then may compute the vector from the upper arch landmark position to the lower arch landmark position, and finally projects this vector onto the lower arch to yield a quantification (e.g., as a scalar) of the amount of delta in “arch1-axis” position there is. This OM may compute how far forward or behind the tooth is positioned on the 1-axis relative to the tooth or teeth of interest in the opposing arch.

Crossbite—Fossa in at least one upper molar may be located by finding the halfway point between distal and mesial marginal ridge saddles of the tooth. A lower molar cusp may lie between the marginal ridges of the corresponding upper molar. This OM may compute a vector from the upper molar fossa midpoint to the lower molar cusp. This vector may be projected onto the d-axis of the archform, yielding a lateral measure of distance from the cusp to the fossa. This distance may define the crossbite magnitude.

Edge Alignment—This OM may identify the leftmost and rightmost edges of a tooth and may identify the same for that tooth's neighbor.
The OM may then draw a vector from the leftmost edge of the tooth to the leftmost edge of the tooth's neighbor.
The OM may then draw a vector from the rightmost edge of the tooth to the rightmost edge of the tooth's neighbor.
The OM may then calculates the linear fit error between the two vectors.
Such a calculation may involve making two vectors:
Vec_tooth=right_tooths_leftside to left_tooths_leftside
Vec_neighbor=right_tooths_rightside to left_tooths_leftside
And then may involve computing the dot-product of these two vectors and subtracting the result from 1. (i.e., EdgeAlignment score=1−abs(dot(Vec_tooth, Vec_neighbor))).
A score of 0 may indicate perfect alignment. A score of 1 may mean perpendicular alignment.

Incisor Interarch Contact KDE—May identify the deviation of the IncisorInterarchContact from the mean of a modeled distribution of such statistics across a dataset of one or more other patient cases.

Leveling—May compute a measure of leveling between a tooth and its neighbor. This OM may calculate the difference in height between two or more neighboring teeth. For molars, this OM may use the midpoint between the mesial and distal saddle ridges as the height of the molar. For non-molar teeth, this OM may use the length of the crown from gums to tip. In some implementations, the tip may be the origin of the local coordinate space of the tooth. Other implementations may place the origin in other locations. A simple subtraction between the heights of neighboring teeth may yield the leveling delta between the teeth (e.g., by comparing Z components).

Midline—May compute the position of the midline for the upper incisors and/or the lower incisors, and then may compute the distance between them.

Molar Interarch Contact KDE—May compute a molar interarch contact score (i.e., a collision depth or other type of collision), and then may identify where that score lies in a pre-defined KDE (distribution) built from representative cases.

Occlusal Contacts—For a particular tooth from the arch, this OM may identify one or more landmarks (e.g., mesial cusp, or central cusp, etc.). Get the tooth transform for that tooth. For each cusp on the current tooth, the cusp may be scored according to how well the cusp contacts the neighboring (corresponding) tooth in the opposite arch. A vector may be found from the cusp of the tooth in question to the vertical intersection point in the corresponding tooth of the opposing arch. The distance and/or direction (i.e., up or down) to the opposing arch may be computed. A list may be returned that contains the resulting signed distances, one for each cusp on the tooth in question.

Overbite—The upper and lower central incisors may be compared along the z-axis. The difference along the z-axis may be used as the overbite score.

Overjet—The upper and lower central incisors may be compared along the y-axis. The difference along the y-axis may be used as the overjet score.

Molar Interarch Contact—May calculate the contact score between molars and may use collision measurement(s) (such as collision depth).

Root Movement d—The tooth transforms for an initial state and a next state may be received. The archform axes at a point L along the archform may be computed. This OM may return a distance moved along the d-axis. This may be accomplished by projecting the root pivot point onto the d-axis.

Root Movement 1—The tooth transforms for an initial state and a next state may be received. The archform axes at a point L along the archform may be computed. This OM may return a distance moved along the 1-axis. This may be accomplished by projecting the root pivot point onto the 1-axis.

Spacing—May compute the spacing between each tooth and its neighbor. The transforms and meshes for the arch may be received. The left and right edges of each tooth mesh may be computed. One or more points of interest may be transformed from local coordinates into the global arch coordinate frame. The spacing may be computed in a plane (e.g., the XY plane) between each tooth and its neighbor to the “left”. May return an array of one or more Euclidean distances (e.g., such as in the XY plane) which may represent the spacing between each tooth and its neighbor to the left.

Torque—May compute torque (i.e., rotation around and axis, such as the x-axis). For one or more teeth, one or more rotations may be converted from Euler angles into one or more rotation matrices. A component (such as a x-component) of the rotations may be extracted and converted back into Euler angles. This x-component may be interpreted as the torque for a tooth. A list may be returned which contains the torque for one or more teeth and may be indexed by the UNS number of the tooth.

Some techniques of the present disclosure, for example the setups comparison technique, and the setups prediction techniques (e.g., such as GDL Setups, MLP Setups, VAE Setups and the like), may benefit from a processing step which may align (or register) arches of teeth (e.g., where a tooth may be represented by a 3D point cloud, or some other type of 3D representation described herein). Such a processing setup may, for example, be used to register a ground truth setup arch from a patient case with the maloccluded arch from that same case, before these mal and ground truth setup arches are used to train a setups prediction neural network model. Such a step may aid in loss calculation, because the predicted arch (e.g., an arch outputted by a generator) may be in better alignment with the ground truth setup arch, a condition which may facilitate the calculation of reconstruction loss, representation loss, L1 loss, L2 loss, MSE loss and/or other kinds of losses described herein. In some implementations, an iterative closest point (ICP) technique may be used for such registration. ICP may minimize the squared errors between corresponding entities, such as 3D representations. In some implementations, linear least squares calculations may be performed. In some implementations, non-linear least squares calculations may be performed. Various registration models may incorporate portions of the following algorithms, in whole or in part: Levenberg-Marquardt ICP, Least Square Rigid transformation, Robust Rigid transformation, random sample consensus (RANSAC) ICP, K-means based RANSAC ICP and Generalized ICP (GICP). Registration may, in some instances, help decrease the subjectivity and/or randomness that may, in some instances, occur in reference ground truth setup designs which have been designed by technicians (i.e., two technicians may produce different but valid final setups outputs for the same case) or by other optimization techniques.

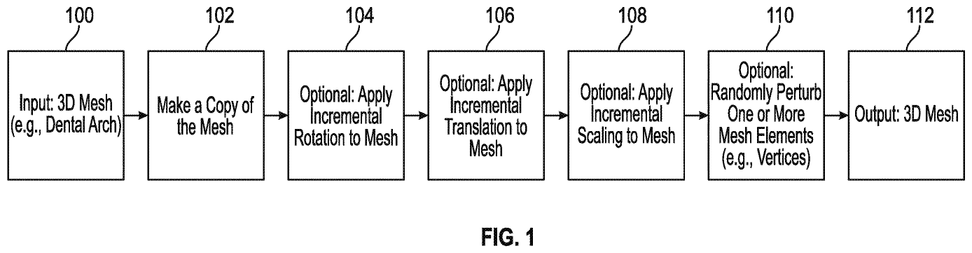

The force-directed graphs methods of this disclosure may draw benefits from data augmentation. Examples include models of this which are trained or evaluated on 3D meshes, such as GDL Setups, RL Setups, VAE Setups, Capsule Setups, MLP Setups, Diffusion Setups, PT Setups, Similarity Setups, FDG Setups, Setups Classification, Setups Comparison, VAE Mesh Element Labeling, MAE Mesh In-filling, Mesh Reconstruction VAE, and Validation Using Autoencoders. Data augmentation, such as by way of the method shown in FIG. 1, may increase the size of the training or evaluation dataset of dental arches. Data augmentation can provide additional training examples by adding random rotations, translations, and/or rescaling to copies of existing dental arches. In some implementations of the techniques of this disclosure, data augmentation may be carried out by perturbing or jittering the vertices of the mesh, in a manner similar to that described in (“Equidistant and Uniform Data Augmentation for 3D Objects”, IEEE Access, Digital Object Identifier 10.1109/ACCESS.2021.3138162). The position of a vertex may be perturbed through the addition of Gaussian noise, for example with zero mean, and 0.1 standard deviation. Other mean and standard deviation values are possible in accordance with the techniques of this disclosure.

FIG. 1 shows a data augmentation method that systems of this disclosure may apply to 3D oral care representations. A non-limiting example of a 3D oral care representation is a tooth mesh or a set of tooth meshes. Tooth data 100 (e.g., 3D meshes) are received at the input. The systems of this disclosure may generate copies of the tooth data 100 (102). In the example of FIG. 1, the systems of this disclosure may apply one or more stochastic rotations to the tooth data 100 (104). In the example of FIG. 1, the systems of this disclosure may apply stochastic translations to the tooth data 100 (106). The systems of this disclosure may apply stochastic scaling operations to the tooth data 100 (108). The systems of this disclosure may apply stochastic perturbations to one or more mesh elements of the tooth data 100 (110). The systems of this disclosure may output augmented tooth data 112 that are formed by way of the method of FIG. 1.

Some techniques of this disclosure may involve the training and/or deployment of neural networks. A 3D representation may be produced using a 3D scanner, such as an intraoral scanner, a computerized tomography (CT) scanner, ultrasound scanner, a magnetic resonance imaging (MRI) machine or a mobile device which is enabled to perform stereophotogrammetry. A 3D representation may describe the shape and/or structure of a subject. A 3D representation may include one or more 3D mesh, 3D point cloud, and/or a 3D voxelized representation, among others. A 3D mesh includes edges, vertices, or faces. Though interrelated in some instances, these three types of data are distinct. The vertices are the points in 3D space that define the boundaries of the mesh. These points would alternatively be described as a point cloud but for the additional information about how the points are connected to each other, as described by the edges. An edge is described by two points and can also be referred to as a line segment. A face is described by a number of edges and vertices. For instance, in the case of a triangle mesh, a face comprises three vertices, where the vertices are interconnected to form three contiguous edges. Some meshes may contain degenerate elements, such as non-manifold mesh elements, which may be removed, to the benefit of later processing. Other mesh pre-processing operations are possible in accordance with aspects of this disclosure. 3D meshes are commonly formed using triangles, but may in other implementations be formed using quadrilaterals, pentagons, or some other n-sided polygon. In some implementations, a 3D mesh may be converted to one or more voxelized geometries (i.e., comprising voxels), such as in the case that sparse processing is performed. The techniques of this disclosure which operate on 3D meshes may receive as input one or more tooth meshes (e.g., arranged in one or more dental arches). Each of these meshes may undergo pre-processing before being input to the predictive architecture (e.g., including at least one of an encoder, decoder, pyramid encoder-decoder and U-Net). This pre-processing may include the conversion of the mesh into lists of mesh elements, such as vertices, edges, faces or in the case of sparse processing—voxels. For the chosen mesh element type or types, (e.g., vertices), feature vectors may be generated. In some examples, one feature vector is generated per vertex of the mesh. Each feature vector may contain a combination of spatial and/or structural features, as specified in the following table:

TABLE 1

| Element | Spatial Features | Structural Features |
|---|---|---|
| Edges | XYZ position of an edge | Edge curvature (depends on a |
| | midpoint, XYZ positions of the | connectivity neighborhood, |
| | edge vertices, or the normal | average curvature of two |
| | vector at an edge midpoint | vertices), dihedral angles, edge |
| | (average of the normal vectors | length, density measure such as |
| | of two vertices). | a count of incident edges (i.e., a |
| | | count of the other neighboring |
| | | edges which share the vertices |
| | | of that edge). |
| Faces | XYZ position of a face centroid, | Face curvature (average |
| | surface normal vector. | curvature of the vertices of the |
| | | face), face area, density measure |
| | | such as count of adjacent faces |
| | | (i.e., which share at least one |
| | | edge with the face). |
| Vertices | XYZ position, normal vector | Vertex curvature, density |
| | (weighted average of the normal | measure such as the count of |
| | vectors of the connecting faces | vertices within a radius of the |
| | for the vertex). | vertex, density measure such as |
| | | the count of incident edges or |
| | | faces. |
| Points | XYZ position | density measure such as the |
| | | count of neighboring points |
| | | within a radius of the point |
| Voxels | XYZ centroid. | Volume, [height × depth × width] |
| | | dimensions, density measure |
| | | such as a count of contained |
| | | vertices, density measure such as |
| | | count of intersected faces, |
| | | density measure such as count of |
| | | intersected edges. |

Table 1 discloses non-limiting examples of mesh element features. In some implementations, color (or other visual cues/identifiers) may be considered as a mesh element feature in addition to the spatial or structural mesh element features described in Table 1. As used herein (e.g., in Table 1), a point differs from a vertex in that a point is part of a 3D point cloud, whereas a vertex is part of a 3D mesh and may have incident faces or edges. A dihedral angle (which may be expressed in either radians or degrees) may be computed as the angle (e.g., a signed angle) between two connected faces (e.g., two faces which are connected along an edge). A sign on a dihedral angle may reveal information about the convexity or concavity of a mesh surface. For example, a positively signed angle may, in some implementations, indicate a convex surface. Furthermore, a negatively signed angle may, in some implementations, indicate a concave surface. To calculate the principal curvature of a mesh vertex, directional curvatures may first be calculated to each adjacent vertex around the vertex. These directional curvatures may be sorted in circular order (e.g., 0, 49, 127, 210, 305 degrees) in proximity to the vertex normal vector and may comprise a subsampled version of the complete curvature tensor. Circular order means: sorted in by angle around an axis. The sorted directional curvatures may contribute to a linear system of equations amenable to a closed form solution which may estimate the two principal curvatures and directions, which may characterize the complete curvature tensor. Consistent with Table 1, a voxel may also have features which are computed as the aggregates of the other mesh elements (e.g., vertices, edges and faces) which either intersect the voxel or, in some implementations, are predominantly or fully contained within the voxel. Rotating the mesh may not change structural features but may change spatial features. And, as described elsewhere in this disclosure, the term “mesh” should be considered in a non-limiting sense to be inclusive of 3D mesh, 3D point cloud and 3D voxelized representation. In some implementations, apart from mesh element features, there are alternative methods of describing the geometry of a mesh, such as 3D keypoints and 3D descriptors. Examples of such 3D keypoints and 3D descriptors are found in “TONIONI A, et al. in ‘Learning to detect good 3D keypoints.’, Int J Comput. Vis. 2018 Vol 0.126, pages 1-20.”. 3D keypoints and 3D descriptors may, in some implementations, describe extrema (either minima or maxima) of the surface of a 3D representation. In some implementations, one or more mesh element features may be computed, at least in part, via deep feature synthesis (DFS), e.g. as described in: J. M. Kanter and K. Veeramachaneni, “Deep feature synthesis: Towards automating data science endeavors,” 2015 IEEE International Conference on Data Science and Advanced Analytics (DSAA), 2015, pp. 1-10, doi: 10.1109/DSAA.2015.7344858.

Various loss calculation techniques are generally applicable to the techniques of this disclosure (e.g., GDL Setups, RL Setups, VAE Setups, Capsule Setups, MLP Setups, Diffusion Setups, PT Setups, Similarity Setups, Setups Classification, Tooth Classification, VAE Mesh Element Labelling, MAE Mesh In-Filling and the imputation of procedure parameters).

These losses include L1 loss, L2 loss, mean squared error (MSE) loss, cross entropy loss, among others. Losses may be computed and used in the training of neural networks, such as multi-layer perceptron's (MLP), U-Net structures, generators and discriminators (e.g., for GANs), autoencoders, variational autoencoders, regularized autoencoders, masked autoencoders, transformer structures, or the like. Some implementations may use either triplet loss or contrastive loss, for example, in the learning of sequences.

Losses may also be used to train encoder structures and decoder structures. A KL-Divergence loss may be used, at least in part, to train one or more of the neural networks of the present disclosure, such as a mesh reconstruction autoencoder or the generator of GDL Setups, which the advantage of imparting Gaussian behavior to the optimization space. This Gaussian behavior may enable a reconstruction autoencoder to produce a better reconstruction (e.g., when a latent vector representation is modified and that modified latent vector is reconstructed using a decoder, the resulting reconstruction is more likely to be a valid instance of the provided representation). There are other techniques for computing losses which may be described elsewhere in this disclosure. Such losses may be based on quantifying the difference between two or more 3D representations.

MSE loss calculation may involve the calculation of an average squared distance between two sets, vectors or datasets. MSE may be generally minimized. MSE may be applicable to a regression problem, where the prediction generated by the neural network or other machine learning model may be a real number. In some implementations, a neural network may be equipped with one or more linear activation units on the output to generate an MSE prediction. Mean absolute error (MAE) loss and mean absolute percentage error (MAPE) loss can also be used in accordance with the techniques of this disclosure.

Cross entropy may, in some implementations, be used to quantify the difference between two or more distributions. Cross entropy loss may, in some implementations, be used to train the neural networks of the present disclosure. Cross entropy loss may, in some implementations, involve comparing a predicted probability to a ground truth probability. Other names of cross entropy loss include “logarithmic loss,” “logistic loss,” and “log loss”. A small cross entropy loss may indicate a better (e.g., more accurate) model. Cross entropy loss may be logarithmic. Cross entropy loss may, in some implementations, be applied to binary classification problems. In some implementations, a neural network may be equipped with a sigmoid activation unit at the output to generate a probability prediction. In the case of multi-class classifications, cross entropy may also be used. In such a case, a neural network trained to make multi-class predictions may, in some implementations, be equipped with one or more softmax activation functions at the output (e.g., where there is one output node for class that is to be predicted). Other loss calculation techniques which may be applied in the training of the neural networks of this disclosure include one or more of: Huber loss, Hinge loss, Categorical hinge loss, cosine similarity, Poisson loss, Logcosh loss, or mean squared logarithmic error loss (MSLE). Other loss calculation methods are described herein and may be applied to the training of any of the neural networks described in the present disclosure.

One or more of the neural networks of the present disclosure may, in some implementations, be trained, at least in part by a loss which is based on at least one of: a Point-wise Mesh Euclidean Distance (PMD) and an Earth Mover's Distance (EMD). Some implementations may incorporate a Hausdorff Distance (HD) calculation into the loss calculation. Computing the Hausdorff distance between two or more 3D representations (such as 3D meshes) may provide one or more technical improvements, in that the HD not only accounts for the distances between two meshes, but also accounts for the way that those meshes are oriented, and the relationship between the mesh shapes in those orientations (or positions or poses). Hausdorff distance may improve the comparison of two or more tooth meshes, such as two or more instances of a tooth mesh which are in different poses (e.g., such as the comparison of predicted setup to ground truth setup which may be performed in the course of computing a loss value for training a setups prediction neural network).

Reconstruction loss may compare a predicted output to a ground truth (or reference) output. Systems of this disclosure may compute reconstruction loss as a combination of L1 loss and MSE loss, as shown in the following line of code: reconstruction_loss=0.5*L1(all_points_target,all_points_predicted)+0.5*MSE(all_points_target,all_points_predicted). all_points_target is a 3D representation (e.g., a 3D mesh or point cloud) corresponding to ground truth data (e.g., a ground truth tooth restoration design, or a ground truth example of some other 3D oral care representation). all_points_predicted is a 3D representation (e.g., a 3D mesh or point cloud) corresponding to generated or predicted data (e.g., a generated tooth restoration design, or a generated example of some other kind of 3D oral care representation). Other implementations of reconstruction loss may additionally (or alternatively) involve L2 loss, mean absolute error (MAE) loss or Huber loss terms.

Techniques of this disclosure may, in some implementations, use PointNet, PointNet++, or derivative neural networks (e.g., networks trained via transfer learning using either PointNet or PointNet++ as a basis for training) to extract local or global neural network features from a 3D point cloud or other 3D representation (e.g., a 3D point cloud describing aspects of the patient's dentition—such as teeth or gums). Techniques of this disclosure may, in some implementations, use U-Nets to extract local or global neural network features from a 3D point cloud or other 3D representation.

3D oral care representations are described herein as such because 3-dimensional representations are currently state of the art. Nevertheless, 3D oral care representations are intended to be used in a non-limiting fashion to encompass any representations of 3-dimensions or higher orders of dimensionality (e.g., 4D, 5D, etc.), and it should be appreciated that machine learning models can be trained using the techniques disclosed herein to operate on representations of higher orders of dimensionality.

In some instances, input data may comprise 3D mesh data, 3D point cloud data, 3D surface data, 3D polyline data, 3D voxel data, or data pertaining to a spline (e.g., control points). An encoder-decoder structure may comprise one or more encoders, or one or more decoders. In some implementations, the encoder may take as input mesh element feature vectors for one or more of the inputted mesh elements. By processing mesh element feature vectors, the encoder is trained in a manner to generate more accurate representations of the input data. For example, the mesh element feature vectors may provide the encoder with more information about the shape and/or structure of the mesh, and therefore the additional information provided allows the encoder to make better-informed decisions and/or generate more-accurate latent representations of the mesh. Examples of encoder-decoder structures include U-Nets, autoencoders or transformers (among others). A representation generation module may comprise one or more encoder-decoder structures (or portions of encoders-decoder structures—such as individual encoders or individual decoders). A representation generation module may generate an information-rich (optionally reduced-dimensionality) representation of the input data, which may be more easily consumed by other generative or discriminative machine learning models.

A U-Net may comprise an encoder, followed by a decoder. The architecture of a U-Net may resemble a U shape. The encoder may extract one or more global neural network features from the input 3D representation, zero or more intermediate-level neural network features, or one or more local neural network features (at the most local level as contrasted with the most global level). The output from each level of the encoder may be passed along to the input of corresponding levels of a decoder (e.g., by way of skip connections). Like the encoder, the decoder may operate on multiple levels of global-to-local neural network features. For instance, the decoder may output a representation of the input data which may contain global, intermediate or local information about the input data. The U-Net may, in some implementations, generate an information-rich (optionally reduced-dimensionality) representation of the input data, which may be more easily consumed by other generative or discriminative machine learning models.

An autoencoder may be configured to encode the input data into a latent form. An autoencoder may train an encoder to reformat the input data into a reduced-dimensionality latent form in between the encoder and the decoder, and then train a decoder to reconstruct the input data from that latent form of the data. A reconstruction error may be computed to quantify the extent to which the reconstructed form of the data differs from the input data. The latent form may, in some implementations, be used as an information-rich reduced-dimensionality representation of the input data which may be more easily consumed by other generative or discriminative machine learning models. In most scenarios, an autoencoder may be trained to input a 3D representation, encode that 3D representation into a latent form (e.g., a latent embedding), and then reconstruct a close facsimile of that input 3D representation as the output.

A transformer may be trained to use self-attention to generate, at least in part, representations of its input. A transformer may encode long-range dependencies (e.g., encode relationships between a large number of inputs). A transformer may comprise an encoder or a decoder. Such an encoder may, in some implementations, operate in a bi-directional fashion or may operate a self-attention mechanism. Such a decoder may, in some implementations, may operate a masked self-attention mechanism, may operate a cross-attention mechanism, or may operate in an auto-regressive manner. The self-attention operations of the transformers described herein may, in some implementations, relate different positions or aspects of an individual 3D oral care representation in order to compute a reduced-dimensionality representation of that 3D oral care representation. The cross-attention operations of the transformers described herein may, in some implementations, mix or combine aspects of two (or more) different 3D oral care representations. The auto-regressive operations of the transformers described herein may, in some implementations, consume previously generated aspects of 3D oral care representations (e.g., previously generated points, point clouds, transforms, etc.) as additional input when generating a new or modified 3D oral care representation. The transformer may, in some implementations, generate a latent form of the input data, which may be used as an information-rich reduced-dimensionality representation of the input data, which may be more easily consumed by other generative or discriminative machine learning models.

In some implementations, an encoder-decoder structure may first be trained as an autoencoder. In deployment, one or more modifications may be made to the latent form of the input data. This modified latent form may then proceed to be reconstructed by the decoder, yielding a reconstructed form of the input data which differs from the input data in one or more intended aspects. Oral care arguments, such as oral care parameters or oral care metrics may be supplied to the encoder, the decoder, or may be used in the modification of the latent form, to influence the encoder-decoder structure in generating a reconstructed form that has desired characteristics (e.g., characteristics which may differ from that of the input data).

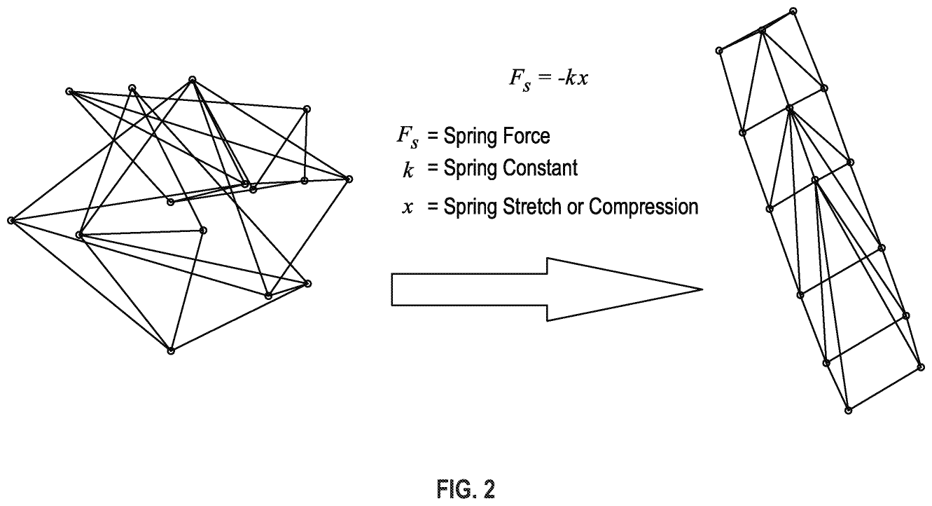

Aspects of this are directed to setups prediction using a force-directed graphs (FDG)-based model. FIG. 2 illustrates a demonstration of an FDG-based setups prediction technique of this disclosure. According to the technique shown in FIG. 2, systems of this disclosure may arrange control points (or nodes) into a target arrangement. FIG. 2 illustrates control points (nodes) that have been placed into a target rectilinear arrangement. In some implementations, the target arrangement may take a form more similar to an arch or a curve and reflect a final setup of teeth for orthodontic treatment.

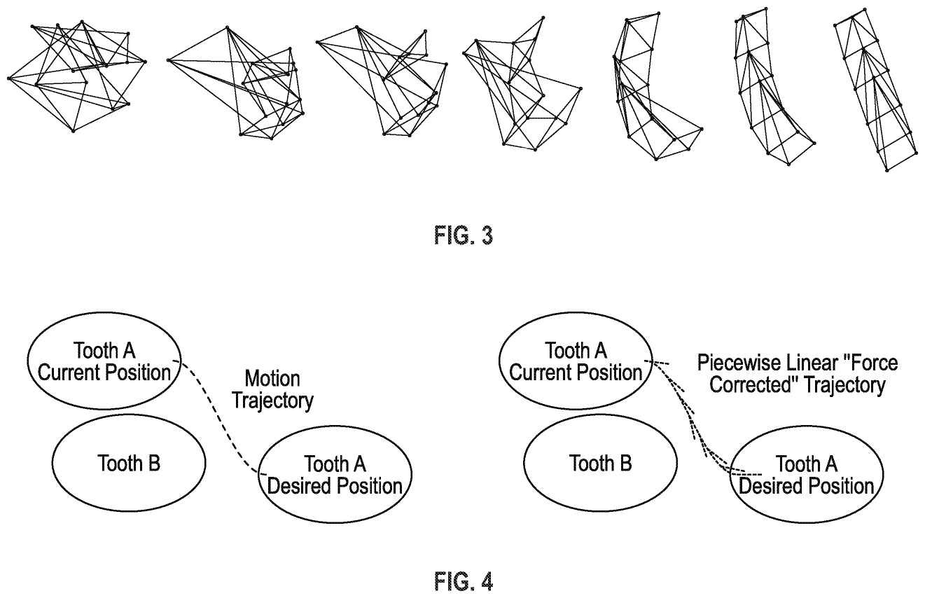

FIG. 3 shows the FDG Setups technique applied to an arrangement of nodes. The nodes of this example are not arranged in a dental setup configuration, but rather, in the form of a proof-of-concept representation that can be applied to the setups problem with minimal modification. For instance, the code corresponding to the FIG. 3 example, with simple modification, can target an arch configuration, rather than the configuration shown in FIG. 3. FIG. 3 shows the progression of control points as the points are moved from an initial arrangement to a final target arrangement.

The nodes may start in a random configuration. Over the course of multiple iterations, and through the application of a force model (e.g., an approximation of a spring-based or another of the forces described herein), the nodes may be arranged into a target structure or pattern (such as that shown on the right side of FIG. 3). In the case of FIG. 3, a repulsion force or “repulsive force” may be defined as a force proportional to a scaled inverse squared distance between nodes.

The FDG-based techniques of this disclosure may use graph optimization and models of physical forces (force-directed graphs) to optimize an arrangement of teeth (a setup) for use in orthodontic treatment. Clear tray aligner (CTA) technology may use a succession of clear plastic trays to move the patient's teeth from their maloccluded poses into poses which meet functional and/or esthetic parameters (e.g., in accordance with patient-provided expectations, doctor-designated procedure parameters, doctor preferences, etc.). Some implementations of the FDG-based techniques determine a final setup (a target or destination arrangement) of the teeth. This technique may also, in some implementations, determine each of a succession of intermediate setups or stages (various levels of “intermediate staging), which represents the intermediate poses that the teeth may assume on the way to the completion of treatment. Some implementations of the FDG-based setups prediction techniques of this disclosure may generate both final setups and intermediate stages, while other implementations may generate either final setups or intermediate stages.

A non-limiting example of the FDG-based setups prediction techniques of this disclosure is described as follows, by way of an analogy to a sponge located in a kitchen area. The sponge may have a predefined shape, which can be expressed as a mesh (or another kind of graph) of the predefined shape. The mesh may be in a stable configuration where the desired edge lengths in the graph make the sponge take on this shape. The edge lengths of the graph may be measured and may be stored. If the sponge is deformed in any way, such as by way of being squeezed by an external agent (e.g., such as a tool, instrument, or hand), the edge lengths between each pair of vertices is distorted as the mesh is deformed in conformance to the sponge deformation. Examination of every node in the deformed-state and further distortion in a way that the edge lengths become slightly closer to their starting lengths represent next steps in the process. Updating these node positions by an incremental amount many times (e.g., tens or hundreds or thousands of times) may eventually bring the sponge back into the sponge's original shape. This is one example implementation of an FDG-based technique.

A dental arch-based use-case example of the FDG-based setups prediction techniques is described below. In this implementation, the use of the FDG is tailored to orthodontic treatment. The input image represents an arch of 32 rigid teeth, and the edge lengths may be represented, at least in part, by the distances between teeth in the setup. The mesh-deformation aspects are analogous to squeezing the teeth (each of which is represented by a node) of the setup into their respective maloccluded poses. Multiple iterations are executed to bring the edge length back to that respective edge's desired length. These multiple iterations may correspond to the stages of orthodontic treatment. This algorithm may also provide a final setup generation, such as by learning a subset of typical distances between teeth in an arch. In some implementations, the positions of the teeth in an arch may be represented as graph vertices, and the FDG-implementing systems of this disclosure may draw an edge between each pair of adjacent vertices to designate relationships between adjacent teeth. In some implementations, these neighbor relationships may be defined between teeth within the same arch (e.g., where an upper left cuspid is a neighbor to an upper left lateral incisor, and an upper left first bicuspid). In some implementations, these neighbor relationships may be defined between teeth of opposing arches (e.g., where an upper left central incisor has a relationship established with the lower left central incisor). In some implementations, the distances incorporate neighboring-tooth relationships that are both intra-arch and inter-arch.

Each edge in the graph may have a maloccluded or “starting” length, according to the maloccluded 3D representation. Each edge in the graph may be designated with a “desired” or target length. Such lengths may be an input to the FDG-based algorithm, learned, or modeled by using a dataset of setups, in some cases using mesh correspondence techniques to identify common, defining features on teeth (e.g. cusps and/or root apexes). Typical distances may be identified between the features on a first tooth and the features of a second tooth in the same arch (or in the opposing arch) and then typical relative positions of these features in typical setups may be learned. A typical relative position may reflect the desired distance between adjacent teeth that may be called for in the final setup or in the intermediate stage.

Various forces that can be used as part of the FDG-based setups prediction techniques are described below. In some implementations, many iterations of the following procedure may be run, where each iteration may move the nodes of the graph in such a way that the edge lengths become slightly closer to their target lengths (e.g., as represented in a final setup). During each iteration, one or more forces may be applied to the nodes of the graph and may move the nodes towards target locations (e.g., as represented in a final setup). These forces may include, but are not limited to, equation-based analogues to Hooke's Law (for springs), Coulomb's Law (for electric charges), and simulated forces which may be proportional to distance (or inversely proportional to distance). Such forces may include analogues to gravity (e.g., modeled using Newton's Law of Universal Gravitation) or nuclear forces, such as the strong nuclear force and the weak nuclear force. In some implementations, such forces may result in attractive or adductive movement between graph nodes. In some implementations, such forces may result in repulsive or abductive movement between graph nodes. Some forces may exhibit either or both of attractive and repulsive forces under different circumstances. Over many iterations, the graph may slowly take on a target arrangement (such as an arrangement that is defined by specified or predicted edge lengths) resulting in either a final setup configuration of teeth or an intermediate stage configuration of teeth. In some implementations, such as in intermediate staging generation, motion limits may be placed on one or more teeth. The advantage of imposing motion limits is to control the displacement applied to one or more teeth for each intermediate stage of orthodontic treatment. In some implementations, each of the forces may have a direction in 3D space and/or a magnitude.

Various physics equations that can be used in the FDG-based setups prediction techniques of this disclosure are described below. In some implementations, the FDG-based setups prediction techniques of this disclosure may use, in whole or in part, elements from Tutte's algorithm (which may, in some implementations, be based on barycentric representations). In some implementations, the FDG-based setups prediction techniques of this disclosure may use, in whole or in part, elements from the Eades layout method or the Frucherman and Rein-gold algorithm, which models nodes as systems of springs and may be based on Hooke's law. In some implementations, the FDG-based setups prediction techniques of this disclosure may use, in whole or in part, elements from Kamada and Kawai's algorithm, which may be also based on a spring model. In some implementations, spring forces may be proportional to the distance between nodes in a setup.

Additional inputs to the FDG-based setups prediction techniques of this disclosure may include one or more of the input values from the vectors from the following list: K, L, M, N, O, R, S, P, Q, U, and V. In some implementations, one or more of the inputs K, L, M, N, O, R, S, P, Q, U, and V may optionally be provided to one or more of the mathematical equations of the FDG-based setups prediction techniques of this disclosure, such as Gauss's Law or Hooke's Law., or an equation for a simulated force which may be either directly or inversely proportional to one or more distance measures, among others. In some implementations, a modified form of a physics equation may be generated by the addition of one or more terms which are derived from one or more of the optional inputs K, L, M, N, O, R, S, P, Q, U, and V. For example, one or more values from one or more orthodontic metrics S may be introduced to Hooke's Law or Gauss's Law to improve the way that teeth are moved through the course of execution of the FDG-based setups prediction techniques. In some examples, one or more metrics S may be provided to an equation which simulates a force which is either directly or inversely proportional to distance.

In some implementations, one or more IPR values (either mesial or distal) may be introduced as terms to one or more of the equations in the FDG-based setups prediction techniques of this disclosure, thereby informing the FDG Setups algorithm about the planned removal of enamel from one or more teeth (which may inform the final and/or intermediate positions of the teeth) and enable collisions to minimized or avoided.

In some implementations, one or more flags from a vector M may indicate that one or more teeth can be fixed, pinned, pontic, extracted, implanted or otherwise warrant special treatment. Fixed teeth may be exempted from movement (e.g., unfixed teeth may be either repelled or attracted to one another, but a fixed tooth may receive a net zero displacement vector and/or zero quaternion and/or identity transform). The FDG-based setups prediction techniques of this disclosure may take as input a specification of the target lengths of the distance between adjacent teeth. The FDG-based setups prediction techniques may compute a set of forces on one or more of tooth nodes and apply displacements (translational and/or rotational) on one or more tooth nodes per time step, and then iterate. In some implementations, information from the procedure parameters K and/or doctor preferences L may influence the calculation of forces which act upon the nodes (e.g., magnitude and/or direction), for example, affecting the magnitude, direction and/or other attributes of the displacement (e.g., to specify which tooth or teeth to apply the displacement to and how much displacement to apply).

Additional implementations of the FDG-based setups prediction techniques of this disclosure are described below. Various considerations listed below pertain to additional non-limiting examples of the FDG-based setups prediction techniques of this disclosure. Defining an equilibrium state of a force system to correspond with the final occlusion or setup arrangement of teeth may provide one or more enhancements to the performance of the FDG-based model. In some implementations, this may entail defining “springs” that have zero net displacement between landmark points on the teeth when the teeth reside in final occlusion. Hooke's law may be applied, wherein zero net displacement may equate to zero net force. In one example, tension-only springs may be used, so that only positive displacements between spring endpoints may be permitted. This model may be particularly useful if the distance between endpoints is always greater than or equal to zero, particularly if the endpoints coincide when the teeth are arranged into a final occlusion. In some cases, however, it may be useful to select endpoints which are displaced in the final occlusion by a distance greater than zero, for example to exert a greater force or higher convergence rate than would otherwise be achieved. Techniques of this disclosure may incorporate this approach to assign greater weight to certain pairs of points than other pairs of points, or to create a bias in the absolute magnitude of force, in effect creating a “packing” force that remains greater than zero even after the teeth have achieved contact and cannot move closer to one another than in their current positions.

In some implementations, a first endpoint can be located on a first tooth, and a second endpoint can be located on a second tooth. An endpoint can be placed or located proximally to at least one oral care landmark, including: a surface of a tooth crown, a surface of a tooth root, a crown centroid, a root centroid, a root center of resistance, a root center of moments, a root center of area, a tooth contact point, an interproximal surface, at least a portion of an appliance (or appliance component), or the like. 3D representations of oral care data may include: a tooth of the patient, a digital pontic tooth, a block of multiple teeth, a crown, a root, a bridge, an implant, a bite block, an orthodontic attachment, a bracket, a button, a restoration, or a portion of cortical bone, among others.

Various overcorrection-related considerations are described below. In some implementations, the techniques of this disclosure may use a bias factor to “overpack” the teeth beyond a geometrically feasible contact arrangement into virtual intersection. The overpacking may be useful for creating so-called “overcorrection stages” that represent physically impossible arrangements of teeth but may enable the creation of orthodontic appliances, such as clear tray aligners, which continue to exert a positive mesio-distal packing force on teeth up to the point of contact. Such a packing force may help to overcome the problem of diminishing forces which may approach zero as the distance between the teeth approaches zero. In biological terms, there may be a minimum threshold force required to cause orthodontic movements of teeth via bone remodeling. This threshold can be overcome for small gaps between teeth by designing an appliance which continues to apply greater than zero force as the gaps close. In terms of a setup design algorithm, an improvement is provided by defining virtual contact points between teeth that are displaced from the true physical contact points, particularly inside the teeth, that result in virtual overpacking of the teeth into slight intersection. Alternatively, greater than zero force may be achieved when the distance between endpoints is zero. Such force may be achieved by changing the force/displacement formula. For example, instead of using F=−kx, one implementation may use F=−kx+x₀, where x₀ simulates a constant displacement from zero, even though there might be no actual displacement between contacting tooth surfaces.

Another possible use for displaced spring endpoints from an ideal coincidence may be to affect the dynamics of convergence during intermediate staging of tooth positions, or in the unrendered iterations of tooth movement used to generate a final occlusion. For example, two contact points in the interproximal region of a pair of neighboring molars which may be selected to coincide in the final occlusion may result in a large shearing force between the teeth when one of the teeth is displaced labio-lingually from the other. This may lead to a rate of tooth movement that is greater than what is biologically feasible when the movement is divided into multiple stages, particularly for the first few stages in the series when the displacement may be large. However, if the spring endpoints are displaced to points inside the teeth, disposed mesially and distally of the contact point in final occlusion, then the force is somewhat tempered when the teeth are in the intermediate stages of movement, because only a subset of the range of forces is exercised by not allowing the force to diminish to zero. In other words, the slope of the force/displacement curve may thereby be reduced. Some implementations of the FDG-based setups prediction techniques of this disclosure may use graph optimization/convergence as a way to define the path of the teeth. From this perspective, the forces may be determined by breaking up the path into piecewise linear segments, thereby allowing the forces associated with each stage to be independently controlled. Stated another way, methods of this disclosure may define a continuous smooth trajectory for the motion of each tooth of the patient's dentition (e.g., when generating a series of intermediate stages for orthodontic treatment). In some implementations, this smooth continuous trajectory (e.g., see “motion trajectory” in FIG. 4) may be divided into multiple discrete steps which collectively approximate that same trajectory. The piecewise linear “force corrected” trajectory on the right side of FIG. 4 is divided into discrete steps, so that individual aligners can be made. The piecewise linear trajectories may, in some implementations, be tangent to the path of motion of a tooth. When moving a tooth from a first pose to a second pose, a larger step may, in some implementations, be defined for certain discrete steps to apply more force during those steps.

Described below are various aspects of local optima that are incorporated into the FDG-based setup prediction techniques of this disclosure. Spring endpoints may be displaced from ideal contact points between teeth for strategic reasons to avoid having teeth get “stuck” in concavities or local minima during staging or setup creation. The systems of this disclosure may select endpoints in a way that the teeth converge on the desired final positions but traverse routes that avoid interference. Two or more springs acting on different tooth landmarks may be used to exploit this effect. By steering each landmark according to its own customized traversal path. In contrast, if only one spring is used per interproximal contact, only one point per neighboring tooth is affected by the spring force, and the remainder of the tooth is either left uncontrolled or affected only by the spring at the opposite end of the tooth. For example, for a pair of neighboring molars, rather than have a single spring between coincident interproximal contacts, the FDG-based techniques of this disclosure may use four springs disposed about the interproximal region, e.g. occlusal-buccal, occlusal-lingual, gingival-lingual, gingival-buccal. By using a plurality of springs (in this particular example, four springs), there may be a greater likelihood, in some situations, that a feature of the tooth surface causing interference can be overcome by virtue of providing a more complex system of forces that change direction and magnitude when contact is made.

In some implementations, unitary springs may be used to provide attractive (and/or repulsive) forces between contact points on teeth, thereby controlling/manipulating the distance between two points. Some implementations, the techniques of this disclosure may exert more granular control by modeling the displacement as a vector X that which may have both a distance component x and a rotational component θ. While F(x) may be analogous to a coil spring placed on an orthodontic archwire between brackets, only able to affect the distance between teeth (actually between brackets), F(θ) may be analogous in one aspect to an archwire able to assert a twisting moment between teeth (along the bracket slot axis). Furthermore, F(X) or F(x, θ) may be used to represent a combination of these devices, affecting both translational and rotational forces between teeth. As such, F(X) may be used to close the space between teeth and to establish a desired relative orientation between the teeth. Alternatively, these forces may be separated as F(x) and F(θ), but may be applied simultaneously, or may be applied sequentially in the same iteration. Without an F(θ) component of the force, the FDG may rely on reaction forces in the tooth roots to achieve the desired third order (torque) orientation between teeth.

According to some implementations of the FDG-based techniques of this disclosure, the operations may be performance based on an assumption that the tooth roots are generally positioned correctly in the alveolar process of the maxilla or mandible, while the crowns are displaced from their correct positions in the dental arch, thus causing malocclusion. In such cases, it may suffice for the FDG to use only a number of springs represented by F(x) between optimal contact points on the crowns of teeth while modeling reaction forces in roots or, more simply, to anchor roots in the alveolar process at center points about which they can pivot. Examples of such center points include a root centroid, a root midpoint along its longitudinal axis, a root apex, a center of moments, etc. In such a system, the torque angles of teeth (the third order rotation of these teeth) may be affected as teeth are forced into alignment by the interproximal springs positioned between the crowns. Because the roots may be constrained about their center points, teeth may have degrees of freedom of movement in all other dimensions until the forces exerted by the springs, or the distances between corresponding contact points, are collectively minimized.

In other cases, where tooth roots may not be positioned correctly in the alveolar process, the FDG-based techniques of this disclosure may correct this circumstance by virtually placing springs between certain landmarks on the roots, such as one or more of the center points listed above, or between points on the roots taken at a common distance from origin points on the crowns. These origin points may be defined to coincide with the occlusal plane when the teeth are properly arranged into a final occlusion, such as on cusp tips or incisal edges, or a given distance from the cusp tip or incisal edge of each tooth that is typically more prominent, such as the cuspids or upper central incisors. Such points may be displaced from these natural landmarks in accordance with one or more orthodontic procedure parameters, like those used to place standardized brackets at prescribed occlusal heights on the teeth. By referencing points on the crowns that ideally align to a common occlusal plane, points on the roots of the teeth may be defined such that all of the root points align to a common root plane. The distances between the crown points and the root points may be varied in the FDG-based techniques of this disclosure in such a way that the second and third order rotation angles of the teeth (namely, angulation and torque) correspond to orthodontic procedure parameters while allowing the crown points to lie along a common occlusal plane and the root points to lie in a common root plane.

In such an arrangement, as long as one or more crown points is/are positioned above or below the mean plane defined by the positions of the totality of the crown points, the outlying crown points may result in their respective tooth or teeth being subjected to some component of force directed toward the mean plane (namely the occlusal plane). Such forces may further serve to level the arch to the plane. A similar effect may occur in the root plane, and as these effects may occur simultaneously in both planes, the teeth may be oriented in accordance with the orthodontic procedure parameters which define a specified distance between the crown points and root points of each tooth. A simple trigonometric formula may define the relationship between each tooth's orientation and the distance between the points of the line segment, e.g. y=d sin θ, where d is the Euclidean distance between points, θ is the angle between a line connecting the points and vertical (normal to the occlusal plane), and y is the distance between points projected onto the occlusal plane (horizontal). The angle θ may be chosen to correspond to an orthodontic second order tip angle (angulation), a third order angle (torque), or a combination of both second and third order angles. In the case of both second and third order angles corresponding to θ, the resultant angle may be computed as a function of both angles applied to the connecting line segment in 3-dimensional space, where the segment projected onto a frontal plane of the tooth may have a tip angle, and the segment projected onto a sagittal plane of the tooth may have a torque angle. The first order rotation of each tooth (orientation about its long axis or a vertical axis) may be controlled by equilibrating spring forces acting on the mesial and distal edges of the crown, e.g. tension springs for which endpoints coincide with interproximal contact points. Because the actual width of the teeth may deviate from expected norms when packed mesio-distally into contact, tip and torque angles may differ to some extent from angles defined by the orthodontic procedure parameters, and/or the crown points and root points might not align precisely with their respective planes when the system of forces reaches equilibrium. This type of reconciliation may occur when applying standardized procedure parameters.

Providing further definition to this arrangement, virtual springs may then be inserted between neighboring teeth such that their endpoints coincide with the root points as defined above. Unlike crown points defined as ideal contact points in the interproximal regions, root points may, in some scenarios, not make contact. In some implementations, the systems of this disclosure may avoid root point contact to prevent injury to the periodontal ligaments. Space may be relatively uniformly distributed between the roots, in an attempt to maximize the efficiency of the use of space while maintaining adequate spacing to avoid root point contact. To accomplish this, the systems of this disclosure may estimate a desired arch length, and compute the space between root points as the arch length divided by the number of neighboring tooth pairs (equal to the number of regions between the roots). The spaces may not be equal in all scenarios, but may be varied according to certain procedure parameter rules or scaled to allow for more space between the centers of teeth with larger root diameters, or teeth having multiple roots. Because the ideal spacing between roots avoids contact, the virtual springs may be defined such that the spring lengths at rest, or equilibrium, are greater than zero. Similarly, the springs are configured to respond to both compression and tension, so that tensile forces may drive the roots closer together, and compressive forces may maintain adequate spacing and/or prevent contact or interference. The techniques of this disclosure may use a different function or force/displacement response curve for tension than for compression or may use a single function that combines terms from different functions, thereby resulting in a composite of responses over different domains. For example, some implementations of this disclosure provide advantages associated with having a repulsive force that approaches infinity as the roots near contact while having an attractive force that is more linear as the roots are separated beyond their ideal spacing. In this way, a maloccluded arrangement of teeth may have many virtual springs under stress, both in tension and in compression (or possibly only under tension between crown points if only tension springs are used), and the teeth may be driven toward a final arrangement that may minimize these stresses, possibly reaching a zero-stress state if there are no interferences preventing the teeth from moving.

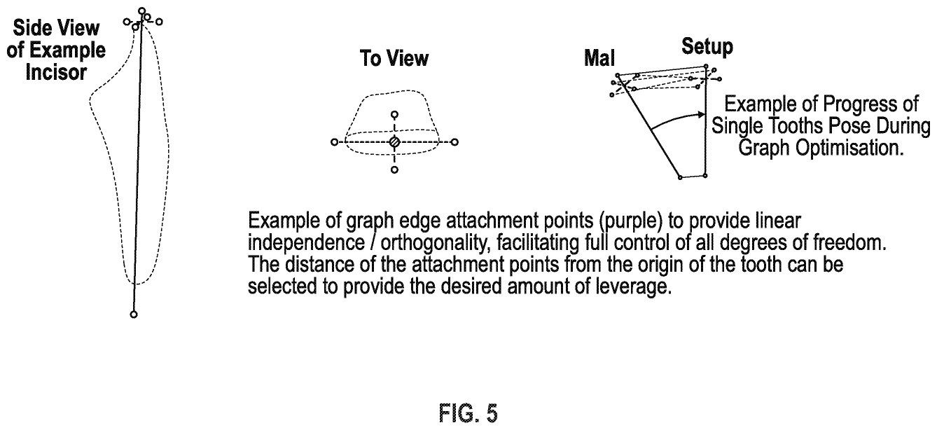

If virtual springs are applied to both the crowns and the roots, it is possible that all degrees of freedom may be adequately controlled without the use of torsion spring forces, as described above. An example of this scenario is expressed by. F(x) without a dependence on θ. In this scenario, tension between the crown points may bring the teeth into contact, effectively forming pivots or rotational axes about a virtual arch form passing through the crowns, and the third order rotation angle about these rotational axes may be controlled by the tension or compression in the root springs, similar to a lever arm. To prevent adverse yaw (first order rotation) of the distalmost teeth, which lack balancing forces from a missing distal neighbor, it may be desirable to use a single, coincident point for both the mesial and distal root springs of each tooth. This point may lie somewhere along the longitudinal axis of the root, or along a best-fit long axis of a plurality of roots on a multi-rooted tooth, such as a bicuspid or molar. By using a single root point, the mesial and distal crown points may fully control the first order rotation of the tooth. Alternatively, discrete mesial and distal root points may be used, but at the risk of competing with the crown points for control of the first order rotation angle. Provided that a sufficient number of points are defined on a tooth, and spatially distributed in such a way as to enable each edge (spring or repulsion, etc.) to control all degrees of freedom, then efficient graph optimization may be enabled, resulting in the graph converging to the predefined stable arrangement. FIG. 5 shows an example of graph edge attachment points (purple) to provide linear independence/orthogonality, facilitating full control of all degrees of freedom. The distance of the attachment points from the origin of the tooth can be selected to provide the desired amount of leverage.

In some implementations, including in combination with implementations described elsewhere in this disclosure, systems of this disclosure may provide technical improvements by using force/displacement functions that differ from the linear function known as Hooke's Law. For example, a constant function F=k may be used in some situations so that F has no dependence on the distance between spring endpoints. In effect, this is the same as F(x)=−kx, where x=1. This formula may have special utility in a dynamic system where the change in distance Δd between spring endpoints may be iteratively recomputed as a function of the current distance between them. By making the force constant, Δd may have the same value for every iteration. In practice, this may be analogous to moving the teeth by fixed increments throughout a series of treatment stages, such as by 0.25 mm of crown displacement prescribed by a clear tray aligner. This may allow for the teeth to advance at a maximum permissible velocity as defined by biological limits, such as the rate of bone remodeling, systolic blood pressure, or a threshold of pain; or by mechanical limits, such as a limit of tray deformation, percent elongation of tray material, a measure of tray fit, or a probability of tray cracking. Stated differently, the systems and techniques of this disclosure more accurately model real-world physiology, which ultimately results in more accurate digital representations and in-turn achieves improved dental or orthodontic treatment outcomes that are performed based upon the more accurate modelling. In some implementations, the FDG-based techniques of this disclosure may use constants only at certain stages of the overall orthodontic treatment, such as mid-treatment when maximum velocity may be achieved reliably, while the beginning and end stages of the treatment may use variable force functions.

In some implementations, the FDG-based techniques of this disclosure may be configured to use non-linear force functions, such as may be used to prevent roots from intersecting, or to accelerate the closure of large gaps until the teeth come within close proximity. In some instances, constant force behavior may be approximated over a range of a non-linear force function that may be linear, parabolic, or exponential. As one example, a logit or inverse sigmoid or inverse hyperbolic tangent (tanh⁻¹x) function may be used to approximate a linear or constant force over a middle range of the function while the extremities of the function's range may be asymptotic to defined values in the domain. Such functions often describe the behavior of super-elastic materials, such as NITINOL or NiTi or other shape memory alloys or polymers. By developing treatment plans based on virtual spring forces that mimic the force/displacement curves of such materials, for example by advancing teeth at a relatively constant pace through the middle stages of treatment and slowing down at the end of treatment or as teeth approach contact with one another, the FDG-based techniques of this disclosure may improve the data precision of the overall orthodontic treatment plan. Slowing the pace of movements at the end of treatment may aid in avoiding interferences which could cause uncertainty in tooth positions, or by allowing more time for lagging teeth to come into conformity with the treatment plan, or by allowing bone density surrounding periodontal ligaments to increase, thereby securing final tooth positions and improving the likelihood of successful retention.

In the same way that root points for virtual springs may increase the degree of control over tooth positions beyond using only one virtual spring per interproximal region (between crowns), greater control may, in some implementations, be achieved using a plurality of corresponding crown points in each interproximal region. As such, springs between root points may be unnecessary, or optional, when a plurality of springs are used between crowns. If collision detection functions are not utilized, all degrees of freedom may be secured by three pairs of crown points per pair of neighboring teeth. If collision detection is used, depending on the shapes of the interproximal surfaces, fewer springs may be used for attractive forces, while colliding tooth surfaces may be used for repulsive forces. Provided that equilibrium is achieved in the system of forces, such that the desired relative positions of neighboring teeth are converged on reliably, any combination of attractive forces and repulsive forces (including collisions) may be used. If only tension springs are used, a dynamic system that reduces the distance between endpoints at every iteration until the distances are zero may cause collisions or intersections between the teeth where convexities in one or both tooth surfaces extend beyond the plane defined by 3 pairs of crown points. If the pairs of points are chosen over broad, flat surfaces, such as between molars, then the interference may be negligible and potentially disregarded in FDG progression. However, if the three pairs of points are chosen over a broad surface between teeth that are more rounded in the interproximals, such as between a bicuspid and a cuspid, or between a cuspid and an incisor, or between two incisors, then the depth of penetration of the sagitta may be greater due to smaller radii of curvature relative to the distance between points (chord length).

In cases in which sagittal penetration is to be avoided, the FDG-based techniques of this disclosure may prevent intersection using collision detection and may allow only tangential contact at the apices of the interproximal surfaces. This point of contact may be treated as a virtual spring having a repulsive force that approaches infinity over an infinitesimally small range of action, or it may be treated as a special case within the program logic of the systems of this disclosure that handles collisions so that any tooth movement that results in intersection is reversed until the intersection is removed. If this contact point was predetermined in the setup that defines the final occlusion as part of the overall orthodontic treatment plan, then the contact point may serve as a constraint on some of the degrees of freedom between the teeth if it's treated as a pair of endpoints for a virtual spring. Some of the FDG-based techniques of this disclosure may prescribe contact at this point to be defined by pairs of virtual spring endpoints radially disposed about the contact point. In terms of spring forces, the contact point may be treated as a spring having little or no attractive force at large distances and a repulsive force that rapidly approaches infinity over a very short activation range, as described above. In effect, this may result in rigid body modeling and prevent intersection. In terms of rigid body mechanics, the contact point may be treated as a fulcrum while one or the other of the teeth may be treated as a lever. If a plurality of virtual tension springs is radially disposed about the contact point, then a balance of forces may be possible such that the tension forces oppose the compression force in the contact. Assuming the contact is modeled by a virtual spring having a force/displacement curve that approaches infinity over a relatively short range of action, then virtually any sum of tension forces may be balanced by an equal and opposite compression force in the contact without appreciable displacement. The above description summarizes the nature of rigid body contacts. One advantage to this type of arrangement may be that the contact point may not be definite, but rather, allows for a sliding contact between teeth that may permit compromise in tooth positions when several neighboring teeth in an arch are competing for space and the plurality of crown point pairs serve better to collectively define the desired tooth relationship than does a single contact point.

Various archwire-related considerations of the FDG-based techniques of this disclosure are described below. A plurality of interproximal contacts per tooth pair may be morphed into something like an orthodontic archwire that exhibits both bending and twisting moments and allows for sliding mechanics. In the above scenario, every crown point in the interproximal region has a corresponding point on the neighboring crown to which the respective crown point aligns in the final occlusion. Bending and twisting moments are possible to a limited degree, as permitted by the range of motion between the crowns when the virtual springs are elongated under tension and optionally constrained by interproximal contact points which prevent intersection. However, an archwire may be simulated by adding layers of these sets of corresponding points in the interproximal region. Rather than having virtual spring endpoints terminate strictly at landmarks on either one tooth or the other in a pair of neighboring teeth, some of the FDG-based implementations of this disclosure may use at least one of terminal springs and intermediate springs. The terminal springs may terminate at one end at landmarks on a tooth and at the other end at intermediate spring endpoints. The intermediate spring endpoints may coincide with other spring endpoints, either from terminal springs or intermediate springs. As such, a series of interconnected springs may be formed. Supporting structure may be added to this arrangement by inserting springs orthogonally and (optionally) diagonally to the above-described terminal and intermediate springs, thereby forming a beam or truss. Each frame of the truss may be formed by a cluster of generally parallel springs (e.g. three springs or four springs) extending from a corresponding number of crown points and braced by as many orthogonal springs and diagonal springs. A series of such frames may be assembled to form an elongated beam or truss. Each frame may be thought of as a structural “element” similar to those used in Finite Element Modeling (FEM) or Finite Element Analysis (FEA), and the macroscopic properties of the assembly may be analogous in some ways to structural beam mechanics when using a material having a finite modulus of elasticity, or Young's modulus, often abbreviated as E, or sometimes Y.

By using this type of arrangement of virtual springs, the FDG-based techniques of this disclosure may guide the teeth along substantially non-linear trajectories from malocclusion to final occlusion. This may help to maintain space between teeth during rotations and thereby avoid blocking interference, collisions between neighboring teeth which may result in premature stoppage of movement before the desired final occlusion is reached. Each series of frames, or elements, may bend and twist in ways that approximate a spline or elastic elongated prism. The prism may be connected to, and terminate at, the interproximal surfaces of neighboring crowns or landmarks beyond the interproximal surfaces, such as cusp tips, gingival margins, facial axis points (FA points), lingual axis points (LA Points), marginal ridge saddle points, etc. Alternatively, the prism may be terminated at a point or plane inside the tooth, such as a crown centroid, a midsagittal plane, or tooth coordinate system origin, a centroid of the gingival margin, etc. This type of termination may allow prisms to be adjoined contiguously into a series of splines or wire-like segments, and to allow for the alignment of teeth according to their native coordinate systems without constraining them to specific, predefined points in the interproximal regions. Adjoining prisms may be aligned such that their ends are mated to common planes, such as the midsagittal planes of the teeth. For example, the distal-most spring endpoints of a rectangular prism between a central incisor and a neighboring lateral incisor might be chosen to coincide with the mesial-most spring endpoints of a rectangular prism between the lateral incisor and its neighboring cuspid. In this way, the prism, or spline, may be made continuous, and its slope may be made continuous in each of three dimensions, thereby transmitting information about each prism's orientation in space to each of its adjoining one or two prisms. Similarly, the curvature of one prism may affect the curvature of its adjoining prism, so that there may be a continuous and gradual change in slope or curvature across the joint. This type of continuity is useful toward avoiding sudden changes in tooth position or orientation as the tooth is optionally moved along the virtual wire or as the wire is relaxed from a high stress or malocclusion state to a low stress or final occlusion state.

The FDG-based techniques of this disclosure may move a tooth virtually along the wire by making stepwise changes to the selection of frame or element that is positioned at the tooth origin or midsagittal plane. The step sizes may be dependent on the mesio-distal length of the frames or elements. Greater resolution and smaller steps may be achieved by using more elements per unit of prism length. Alternatively, intermediate tooth positions may be achieved by using linear interpolation or simply computing an intermediate point along a line defined by the element's endpoints, or by interpolating a position along a spline that models the curvature of a series of elements comprising a prism under stress, possibly deformed by bending and/or twisting moments. Moving a tooth along a curved wire may potentially avoid interference between irregular or protrusive or interlocking tooth features which may cause movement to stop prematurely, before the desired final position is achieved. The curved path may route the tooth around such features instead of driving the tooth straight into the features.

Additional implementations of the FDG-based techniques of this disclosure are described below. In one example implementation, the virtual springs between the nodes of each frame or element are used to model a force/displacement function that contains both compressive and tensile components. As such, each virtual spring may have a positive, non-zero equilibrium length that results in the prism holding an equilibrium shape. The equilibrium shape may be straight or curved. Alternatively, the lengthwise (and optionally, the diagonal) virtual springs extending between the teeth may be modeled as having only tensile forces, while the orthogonal virtual springs comprising the supporting framework may be modeled as having both compressive and tensile forces. As such, the support frames may largely retain their shape, while the lengthwise (and optionally, the diagonal) virtual springs may grow shorter and shorter after each iteration of computation and tooth movement as teeth are progressed from a malocclusion state to a final occlusion state. In this way, a series of interconnected springs between teeth may better serve to route teeth around irregular or protrusive or interlocking tooth features which might cause movement to stop prematurely before the desired final positions are achieved. Collision detection may also be employed to detect and remove intersections between teeth after each iteration, and to determine the resting state of the teeth when stresses remain in the wire. Collision detection aids in those implementations which connect the prismatic segments of wire inside the teeth, such as at the tooth origins or midsagittal planes, because the locations of the interproximal regions may not automatically be identified by the terminal points of the prisms.

In yet another implementation, every tooth may be connected to each of its neighbors by a virtual spring that terminates at the crown centroid or tooth origin, which serves to pack the teeth into contact but not change the orientation of the teeth, while a separate spring acting only on the tooth centroid or origin may be employed to orient the tooth with respect to three degrees of rotation according to one or more procedure parameters. Collision detection functionalities may prevent intersection/overpacking. By having the interproximal springs act on the tooth centroids without applying any moments, the teeth may be allowed to freely rotate independently of any tension or compression forces. Alternatively, the interproximal springs may be substituted with a “thin” wire, similar to the wire described above but with only 1 lengthwise spring per element and no twisting moments. Twisting moments may, in some implementations, not be required to orient the teeth if each tooth has its own 3D spring located at its center to orient the tooth according to one or more procedure parameters. Optionally, first and second order rotation angles may be determined by the slope or direction of the wire at the tooth origin or centroid, while third order rotation angles may be determined according to one or more procedure parameters and expressed in degrees from vertical or degrees from an occlusal plane, for example. In this way, at least a portion of the route taken by the teeth on their paths from malocclusion to final occlusion may be guided by a curved wire and given the possibility of reduced interference by routing the teeth around potential obstructions.

In yet another implementation, instead of connecting springs to a plurality of interproximal contact points, to the FDG-based techniques of this disclosure may employ just one spring per interproximal contact and control the remaining degrees of freedom by connecting the FA point of each tooth to a labial arch form and/or the LA point of each tooth to a lingual arch form. Another optional operation is to connect cusp tips and incisal edges to an occlusal arch form or occlusal plane. Whether using an arch form spline or an occlusal plane, the endpoints of the springs may be allowed to “slide” along the spline or plane without friction. In other words, the iterative method that asserts a stepwise change in distance between the tooth and the spline or plane by moving the tooth may not move the tooth toward a predefined point but instead recompute the nearest point on the spline or plane at every iteration. In effect, the endpoint may be allowed to move or “slide” as the tooth moves while still acting in the direction of the force. Tensile or attractive forces may logically act on the nearest point, because a sliding endpoint may naturally move to minimize the energy stored in the spring. However, a compressive or repulsive force may optionally be designed either to act on the nearest point or the farthest point. The farthest point may be more natural in the respect that a spring under compression wants to expand in length, and a sliding endpoint may move until energy is minimized at the farthest point. Repulsive forces may only result in converging endpoints on curves or curved surfaces having at least one apex or maximum, such as a definite point that is farthest from the other endpoint of the spring. For straight lines or planes, this point is at infinity.

The force-directed graphs techniques of this disclosure may benefit from integration with generative ML methods. For example, an ML model may be trained to generate (or modify) a force-directed graph may generate (or modify) a data structure which describes a force directed graph. Such an ML model is called a force-directed graph generation or modification (FDGGM) ML model. In some implementations, a FDGGM may be trained using representation learning, which may involve at least a first ML module and a second ML module. One or more 3D representations of the patient's dentition (e.g., 3D tooth meshes) may be provided to the first ML module, which may generate one or more latent representations of the patient's dentition. The one or more latent representations may have a lower order of dimensionality than the inputted 3D representations. The one or more latent representations may be provided to a second ML module, which may generate one or more data structures describing one or more force directed graphs corresponding to the patient's dentition.

The first ML module may, in some implementations, comprise one or more U-Nets, one or more 3D SWIM transformers, one or more pyramid encoder-decoders, any of which may compute hierarchical neural network features using the patient's dentition. Furthermore, the first ML module may, in some implementations, comprise one or more encoders (e.g., an encoder that was trained as a part of a reconstruction autoencoder—such as a variational autoencoder with optional normalizing flows), one or more sets of 3D convolution and 3D pooling layers, one or more transformer encoders, or one or more transformer decoders, among other neural network architectures. In some implementations, the 3D representation of the patient's dentition may comprise one or more mesh elements. A mesh feature module may be used to compute, according to the descriptions herein, one or more mesh element features. For example, for each mesh element, a mesh element feature vector may be computed. These mesh element feature vectors may be provided to the first ML module, and improve the accuracy of the resulting latent representation, because the mesh element feature vectors inform the first ML module about aspects of the shape and/or structure of the 3D representation of the patient's dentition which may not otherwise be easily ascertainable by the first ML module.

The second ML module may, in some implementations, comprise one or more multi-layer perceptrons (MLP), one or more encoders, or one or more transformer structures (e.g., a transformer encoder which generates a latent representation which is then reconstructed by a decoder, or a transformer decoder which generates a latent representation which is then reconstructed by a decoder), among others. In some implementations, the second ML module may contain one or more fully connected layers (e.g., 4 layers) with optional skip connections.

In some implementations, two or more intermediate stages (or arrangements) of teeth may be provided to the FDGGM ML model, which may generate one or more data structures describing one or more force directed graphs. The generated one or more data structures may describe force directed graphs which may move the patient's teeth through the two or more intermediate stages which were provided as a part of the training dataset. In this fashion, the FDGGM ML model may be trained to generate one or more force directed graphs which may move the teeth of a patient through incremental steps which correspond to the series of trays used in orthodontic aligner treatment. The predicted one or more data structures may be compared to one or more corresponding data structures (e.g., data structures that describe force directed graphs) which are a part of the training dataset. Loss may be computed as a result of the comparing (e.g., using L1, L2, MSE, or other loss calculation methods described herein), and the computed loss may be used to train, at least in part, one or more of the first ML module or the second ML module. In some implementations, a set of intermediate stages (e.g., 10 stages) may be provided for a patient case in the training dataset, and the FDGGM ML model may predict (or generate) a single force directed graph data structure which will reproduce the series of tooth movements that are shown in the set of intermediate stages. Stated another way, the FDGGM ML model may predict attachment points, torque vectors (or the like), or other aspects of the data structure for a force directed graph for the patient's particular dentition. That data structure may describe a force-directed graph that when executed on the patient's dentition, may generate tooth transforms which are capable of moving the teeth through a series of intermediate stages (or intermediate setups).

In some implementations, a force-directed graph may comprise representations of one or more teeth, and representations of one or more springs (or connections or linkages) between those teeth. A non-limiting example of a force directed graph data structure may contain one or more of the following attributes or data fields.

### For Each Tooth:

Dental Status (e.g., tooth number, tooth type, missing, extracted, fixed, pinned in setup, pontic, block member, etc.)
Tooth Geometry—3D representation of crown surface (e.g., 3D mesh) and optional 3D representation of root surface
Root Length—real number
Root Center of Resistance—real 3D point
Spring Number(s)—integer(s)

### For Each Spring Connecting Tooth a to Tooth B:

Spring Number—integer
Tooth A Number—integer
Tooth A Attachment Point—tuple, e.g. (x, y, z)
Tooth A Attachment Axial Vector—tuple, e.g. (u, v, w)
Tooth A Attachment Torque Vector—tuple, e.g. (u, v, w)
Tooth B Number—integer
Tooth B Attachment Point—tuple, e.g. (x, y, z)
Tooth B Attachment Axial Vector—tuple, e.g. (u, v, w)
Tooth B Attachment Torque Vector—tuple, e.g. (u, v, w)
Spring Constant (or Function)—real number

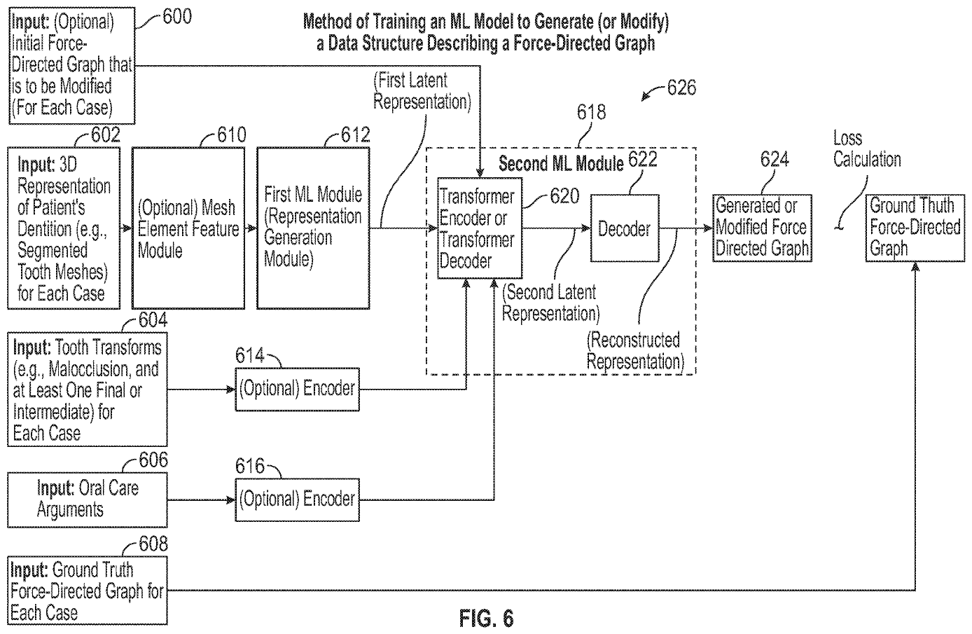

FIG. 6 shows a method of training a machine learning (ML) model to generate a predicted force-directed graph, or to modify an existing force directed graph 600 which is provided as associated with the patient case and that is provided as input (e.g., a partially configured or partially designed force directed graph which is to be completed). When the method 626 is trained to modify an existing force-directed graph for a patient case of the training dataset, an initial or existing (optional) force directed graph 600 may be provided as input. One or more 3D representations 602 of the patient's dentition (e.g., segmented tooth meshes) may be provided to the (optional) mesh element feature module 610, which may compute mesh element feature vectors according to the description herein. The output of the mesh element feature module 610 may be provided to first ML module 612, which may generate one or more first latent representations of the patient's dentition (e.g., teeth). The one or more first latent representations may be provided to the second ML module 618. Tooth transforms 604 may be provided for each patient case. The tooth transforms 604 may include the malocclusion transforms for the teeth, and transforms for one or more subsequent stages (e.g., for at least one intermediate stage or for the final setup). The tooth transforms 604 may, in some implementations, be provided to an encoder 614, which may generate latent representations of the transforms. Oral care arguments 606 may be provided to an optional encoder 616, which may encode the oral care arguments 606 into latent representations. The oral care arguments 606 may be provided to the second ML module 618 (e.g., to a transformer encoder or transformer decoder 620 of the second ML module 618). The second latent representation generated by module 620 may be provided to a decoder 622, which may reconstruct the second latent representation into a predicted data structure 624 which describes a force-directed graph. The predicted data structure 624 may be compared to a corresponding ground truth (or reference) data structure for a force-directed graph (e.g., a force-directed graph which is known to be correctly configured for the generation of intermediate stages for the particular example in the training dataset). One or more losses (e.g., MSE or others described herein) may be computed between the predicted and ground truth data structures. The one or more loss values may be used to train, at least in part, the neural networks of the first ML module and/or the second ML module. For example, the losses may be used to update the neural network weights of a partially train ML model, such as by backpropagation. In other implementations, other neural network architectures may be used inside the second ML module. For example, the second ML module may contain one or more encoders, one or more multi-layer perceptrons, one or more decoders, or the like.

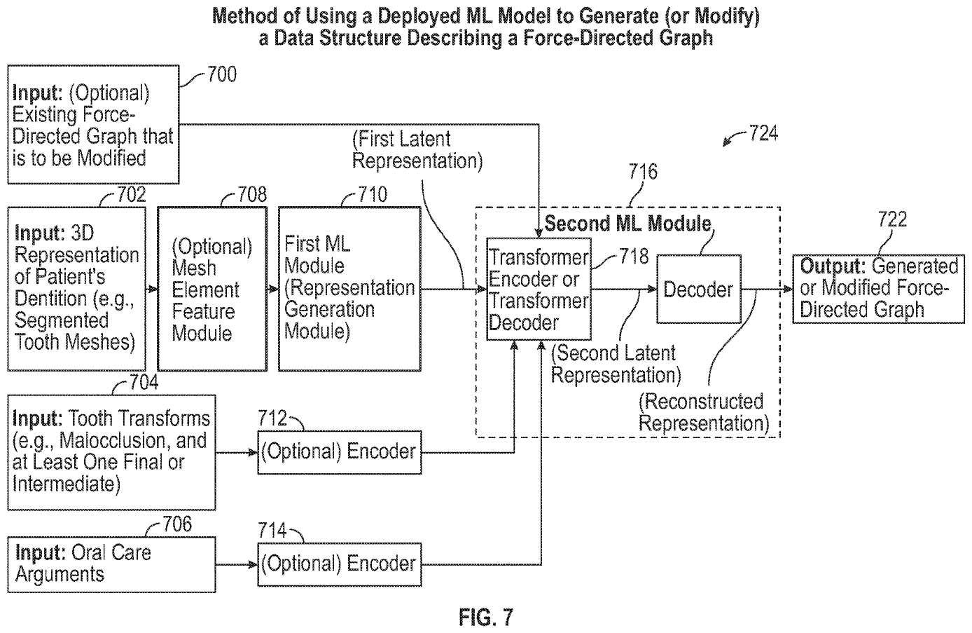

FIG. 7 shows a method 724 of using a fully trained machine learning (ML) model to generate a predicted force-directed graph, or to modify an existing force directed graph 700 that is provided as input (e.g., a partially configured or partially designed force directed graph which is to be completed). When the method 724 is trained to modify an existing force-directed graph for a patient case of the training dataset, an initial or existing (optional) force directed graph 700 may be provided as input. One or more 3D representations 702 of the patient's dentition (e.g., segmented tooth meshes) may be provided to the (optional) mesh element feature module 708, which may compute mesh element feature vectors according to the description herein. The output of the mesh element feature module 708 may be provided to first ML module 710, which may generate one or more first latent representations of the patient's dentition (e.g., teeth). The one or more first latent representations may be provided to the second ML module 716.

Tooth transforms 704 may be provided for the patient case. The tooth transforms 704 may include the malocclusion transforms for the teeth, and transforms for one or more subsequent stages (e.g., for at least one intermediate stage or for the final setup). The tooth transforms 704 may, in some implementations, be provided to an (optional) encoder 712, which may generate latent representations of the transforms. Oral care arguments 706 may be provided to an optional encoder 714, which may encode the oral care arguments 706 into latent representations. The oral care arguments 706 may be provided to the second ML module 716 (e.g., to a transformer encoder or transformer decoder 718 of the second ML module 716). The second latent representation generated by module 718 may be provided to a decoder 720, which may reconstruct the second latent representation into a predicted data structure 722 which describes a predicted force-directed graph.

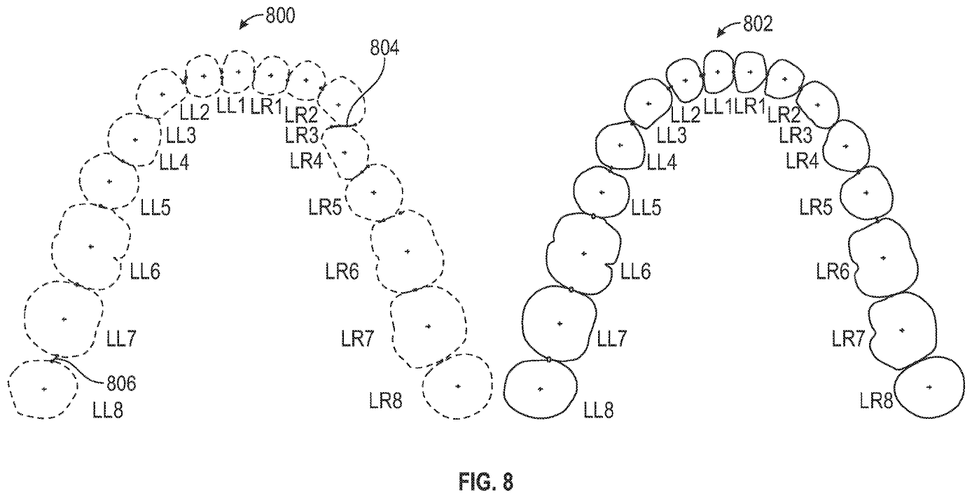

In some implementations, the force-directed graphs-based setups prediction models of this disclosure may be configured to move the one or more maloccluded teeth of the patient (e.g., dentitions commonly have 28 teeth between the upper and lower arches) into final occlusion (or final setups) poses. The methods may attach virtual springs to the teeth of each arch and allow the forces acting through those springs to incrementally move the teeth into final occlusion poses which are suitable for use in oral care appliance generation. The methods may generate a series of intermediate stage poses of the teeth by computing, for each tooth, a vector sum of the forces and moments exerted on each tooth by the one or more springs attached to it, doing this for all of the teeth permitted to move in the setup (non-fixed), then moving each tooth in a direction and by a distance proportional to the net forces and moments acting on it. In general, forces result in net translations, and moments result in net rotations of the teeth. The combined effect for each tooth is represented as a 3D Affine transform, which results in a motion that may include both a translation and a rotation simultaneously. After all of the teeth have been moved, the process is repeated in a subsequent iteration of computing forces and moments and moving teeth, until forces and moments have become sufficiently diminished or tooth movements have slowed to a minimum threshold of displacement per iteration. Note that tooth collisions may be detected during each iteration, preferably before or as part of the forces and moments computation, which may serve to alter the directions and magnitudes of the net forces and moments. The effect of these collisions is to simulate rigid body mechanics, which prevents geometric solids from intersecting. The methods may also generate final occlusion poses for the teeth, after the completion of the last intermediate stage. The methods may operate on 3D representations of the patient's teeth (e.g., 3D meshes). One or more virtual springs may be attached between pairs of teeth in an arch (e.g., between immediately adjacent teeth, among others). In some implementations, these virtual springs may be attached according to rules described herein. In some implementations, a fully trained ML model 724 may be used to configure a force directed graph, such as by generating attachment points for one or more virtual springs 804 and 806 (among the several virtual springs drawn between teeth in FIG. 8) between pairs of teeth, among other operations.

FIG. 8 shows a 2D cross section of the 3D tooth meshes. Although this diagram is depicted in 2D, it should be understood that the force-directed graphs techniques of this disclosure are operable to function in either the 2D or 3D domains. The arch 800 shows a patient's teeth in maloccluded poses. Virtual springs 804 and 806 (among others) are placed to connect adjacent teeth. In some implementations, a trained ML model may generate the attachment points for the virtual springs, among other aspects of the force directed graph. The arch 802 shows the patient's teeth in final occlusion (e.g., final setup), as the teeth appear at the end of treatment using a force-directed graph setup model.

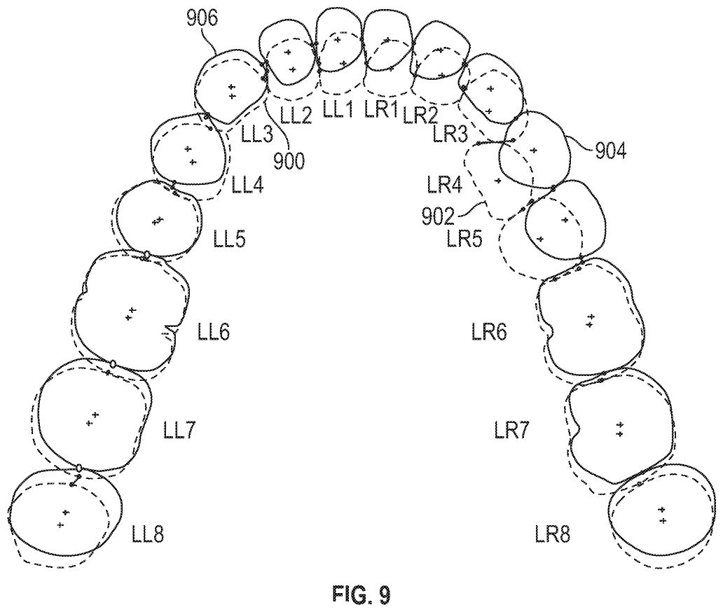

FIG. 9 shows the arch 800 superimposed with the arch 802. The tooth LL3 is shown in malocclusion 900 and in final occlusion 906. The tooth LR4 is shown in malocclusion 902 and in final occlusion 904.

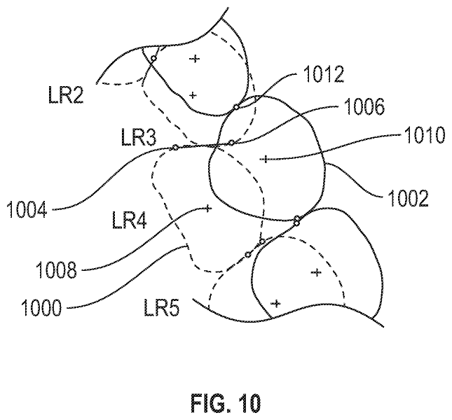

FIG. 10 shows an area of interest in FIG. 9 that illustrates attachment points and springs connecting those attachment points. The tooth LR4 is shown in malocclusion 1000. The tooth LR4 is shown in final occlusion 1002. Attachment point 1004 on the maloccluded tooth LR4 connects via a virtual spring to attachment point 1006 on the maloccluded tooth LR3. In the final occlusion, points 1004 and 1006 are on top of each other 1012. In some implementations, a virtual spring (not shown) may be connected between tooth centroids 1008 and 1010.

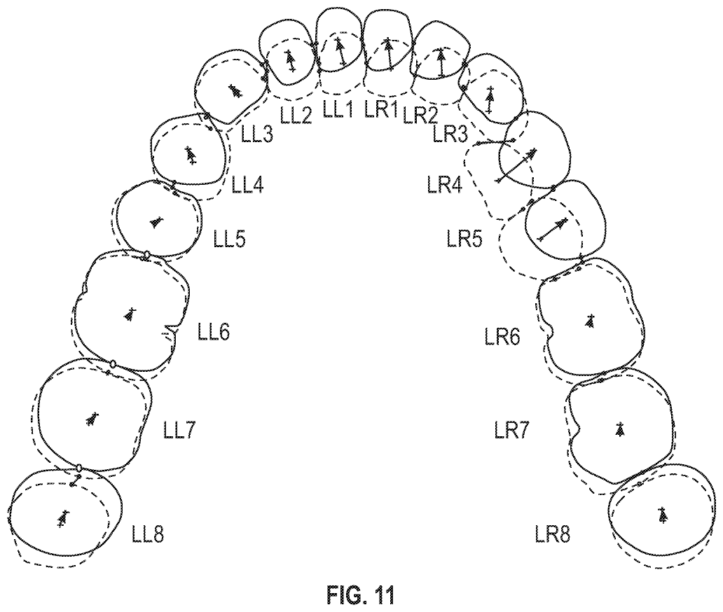

FIG. 11 shows the arch 800 superimposed with the arch 802, where vectors show the direction of motion of the tooth centroids. In some implementations, virtual springs may be connected between the tooth centroids, or other landmarks, to move the teeth from malocclusion to final occlusion. In such implementations, rather than connect neighboring teeth in an arch to one another, which can be useful in determining a final occlusion when none has yet been defined, identical teeth may be connected to themselves in 2 different poses or stages of treatment, such as connecting teeth in their malocclusion positions to congruent representations of the same teeth in their final occlusion positions. Although this approach requires the final positions of the teeth to be predetermined by other means, such as other implementations described herein, it can be useful for determining the intermediate positions or stages of the teeth mid-treatment. Such intermediate positions or stages of treatment may be analogous to Clear Tray Aligners (CTAs) or custom archwires or other removable appliances that could be changed periodically during treatment and customized to assert movements of the teeth according to a prescribed treatment plan. Such implementations may have the advantage of guiding the teeth toward their target positions while resolving collisions between them. When teeth are treated as rigid bodies or geometric solids, the computation of net force vectors results in directional changes as teeth collide. This prevents or resolves intersections of neighboring teeth and causes the directions of motion to change in such a way that intersections are avoided in subsequent iterations of movement. In effect, teeth are forced to detour around one another, rather than intersect one another. This type of motion planning is advantageous over mere linear interpolation of individual tooth transforms from malocclusion to final occlusion (or target arrangement to target arrangement) because linear interpolation may result in intersections of neighboring teeth. A force directed graph approach can have the effect of negotiating movements of the teeth by giving higher priority to teeth which are further from their targets, because their springs are further deflected from equilibrium and therefore result in higher tensile forces. These forces translate into proportional step sizes as the teeth are moved from stage to stage in each iteration of computation. Note, of course, that each iteration of movement need not coincide with an appliance. There can be many small iterations of movement for each appliance to simulate the paths taken by the teeth to avoid collision.

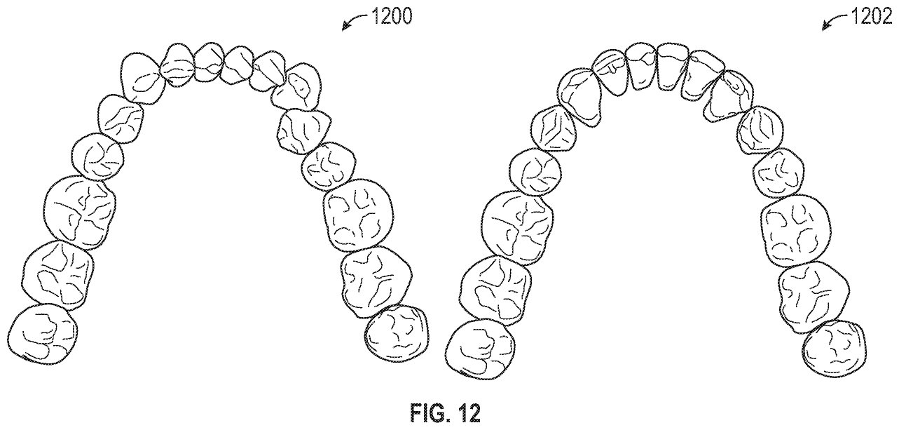

FIG. 12 shows 3D tooth meshes 1200 in malocclusion and 3D tooth meshes 1202 in final occlusion (e.g., final setup).

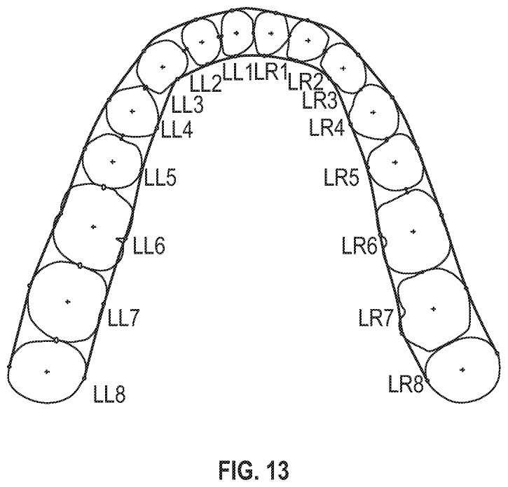

In the example in FIG. 13, each tooth has 3 attachment points, and each interproximal has 3 springs. All 3 attachment points are on the surface of the tooth: the interproximal near the contact point in the final occlusion, the Facial Axis (FA) Point (center of the facial surface of the crown), and the Lingual Axis (LA) Point (center of the lingual surface of the crown). By using 3 attachment points, all degrees of freedom are controlled. The further apart the attachment points, the greater the leverage and thus the greater the possibility of controlling each tooth. In this example, all 3 attachment points are well distributed within a horizontal (or occlusal) plane, but they are generally concentrated around this plane with regard to their vertical disposition. Greater leverage in other dimensions, such as torque (3rd order rotation) can be achieved by placing some of the points on the tooth roots, such as the root centroids or root apices or root centers of resistance, instead of the lingual surfaces, for example, or in addition to all of the existing attachment points (using either 3 or 4 attachment points per tooth). Note also that in this example, each of the labial and lingual attachment points for each tooth are shared between the mesial and distal springs, i.e. the mesial and distal attachment points are coincident. However, this is not strictly necessary. The mesial and distal attachment points may be distinct and positioned closer to their respective mesial or distal surfaces of the teeth, as is the case with the interproximal attachment points near the tooth contacts. In some implementations, other attachment points may be defined instead of or in addition to the ones shown, such as 3 or 4 attachment points (and corresponding springs) distributed radially about the contact point on the mesial or distal surface of each tooth, analogous to a ligament or other connective tissue. In this case, the springs being closer to the contact surfaces would be shorter in their equilibrium states and perhaps offer greater control in certain situations, such as when teeth are in relatively close proximity to begin with. However, in other situations, such as when there is a combination of significant shear strain and first order rotation between neighboring teeth, it might be better to use labial and lingual attachment points to overcome local gridlock that could otherwise occur if a convex surface of a tooth cannot be overcome by lateral spring forces. Such choices of where best to place attachment points and springs may be determined by a neural network that learns the consequences of each placement as a function of the malocclusion and final occlusion for a given case after a series of intermediate tooth positions or stages is determined. A reinforcement learning method may be used, for example, to reward placements that result in a final occlusion being reached with little or no interference, and to punish placements that result in interference, stoppage of movement, gridlock, or prolonged movements. Note also that different spring forces may be used as means to different ends in various scenarios. For example, the springs between labial or lingual attachment points may be defined to be at rest in the final occlusion when their lengths are greater than zero, as shown above. However, if additional force is needed to overcome obstacles or interferences, it may be advantageous to define their rest lengths to be shorter than the distances between attachment points in the final occlusion, thus leaving tension in the final setup. To prevent further movement in wrong directions, these spring forces must be balanced on opposite sides of each tooth, so that a system of forces created by a plurality of springs achieves a net zero force when the tooth is in the desired final position. To the same end, it can also be advantageous to bias the force exerted by a spring to be greater or less than a proportionality constant multiplied by its length, i.e. instead of F=−kx, use F=−kx+F₀, where F₀ is a non-zero force that persists even when the displacement x of the spring equals zero. This can be used to overcome diminishing forces as teeth get very close together and to avoid teeth getting “stuck” on little bumps or surface features or to overcome static friction during minute, lateral movements (parallel to the contact plane or in shear along the contact surfaces).

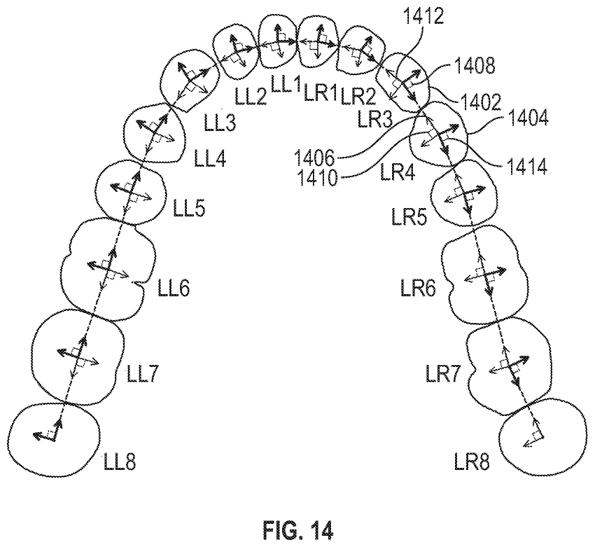

FIG. 14 shows an arch of teeth in a final or setup stage of orthodontic treatment, along with springs connecting adjacent teeth at their centroids, wherein each endpoint of each spring is assigned a coordinate system comprising a set of 3 mutually perpendicular vectors, one being oriented generally in a mesio-distal direction (axial to the spring), another being oriented in a generally labio-lingual direction, and another being oriented generally in an occluso-gingival direction. In particular, the coordinate systems of each pair of endpoints are defined such that the distance between them is equal to the length of the spring at rest, the mesio-distal vectors are colinear and the labio-lingual vectors are parallel (i.e. the spring is not bent), and the occluso-gingival vectors are parallel (i.e. the spring is not twisted). In other words, in this example, the springs are relaxed, and the system is at rest. As one example, tooth LR3 1402 is adjoined by both a mesial spring and a distal spring 1406 and defines coordinate systems 1412 and 1408, respectively, for one endpoint of each. Similarly, tooth LR4 1404 defines coordinate systems 1410 and 1414 for each of a mesial and a distal spring, respectively. As a whole, the set of springs, the spring endpoints, the coordinate systems of the endpoints, and any deflections of the endpoints from their equilibrium positions & orientations (in a stressed state) comprise a Force Directed Graph (FDG). In particular, as it pertains to Graph Theory, the springs may be analogous to arcs, and the spring endpoints or coordinate systems or teeth may be thought of as nodes. In some embodiments, some or all or none of the spring endpoints or coordinate systems or teeth may coincide as nodes in the graph. In some embodiments, a node may comprise a tooth while a coordinate system is an additional data element associated with a node. In some embodiments, 1 or 2 or more springs may share an endpoint and a coordinate system, while in other embodiments, 1 or 2 or more springs may share an endpoint but associate different coordinate systems with each endpoint. In other embodiments, an endpoint may be analogous to a node, while a tooth is an associated data element. In other embodiments, contact points between teeth may serve as temporary nodes that are added and removed from the graph as contacts might occasionally occur and resolve during a simulated course of treatment. In some embodiments, a node or tooth or other 3D data element interacting with the dentition may associate a resultant force and/or moment comprising a sum of 2 or more forces and/or moments acting on said tooth or 3D data element, or a point thereon or therein. In further embodiments, the resultant force associated with said node or tooth or other 3D data element may be used to compute and further associate one or more displacements of the node or tooth or 3D data element, such as a translation, a rotation, a transformation (transform), or a deformation (compression, tension, shear, scale, shape change, material addition, material subtraction, etc).

In the example of FIG. 14, all degrees of freedom for each tooth may be controlled using only 1 spring per interproximal region, or as shown herein, 1 spring per intercentroid region. To accomplish this, each spring endpoint associates with at least a pair of orthogonal vectors, thereby constituting a coordinate system that defines the orientation of the spring at its endpoint. The coordinate system is defined by an axial vector, which is directed generally (although not necessarily) along the axis of the spring toward the opposite endpoint, and a torque vector, which is directed orthogonal (perpendicular) to the axial vector, i.e. labially, lingually, occlusally, gingivally, etc. In addition to compression or tension forces along the axis of the spring, these additional coordinate systems, each defined by a pair of vectors and an endpoint, in effect allow for bending and twisting moments in the spring. In addition to axial forces which primarily control the distance between neighboring teeth via compression or tension in the springs, these additional moments serve to control the orientations of the teeth, e.g. 1st order rotation (“rotation”), 2nd order rotation (“tip” or “angulation”), and 3rd order rotation (“torque”). The coordinate system may be thought of as a spherical spring, or a system of 3 rotary springs, that may be deflected about 1 or 2 or 3 axes simultaneously and have a tendency to return to its equilibrium orientation when relaxed. Similarly, a pair of these coordinate systems, one at each end of a spring connecting 2 teeth, may be thought of as a spring capable of bending and twisting moments which change the orientations of the endpoints. The endpoints have a tendency to return to their equilibrium orientations as the spring relaxes. Each orthogonal dimension of the coordinate system at an endpoint may independently define a spring torque function, such as Tₓ=−kₓα, T_{y}=−k_{y}β, and T_{z}=−k_{z}γ, where α, β, and γ are rotation angles about the x, y, and z axes, respectively, of the coordinate system of an endpoint. Given the said torque functions, each torque may be proportional to the deflection from the equilibrium orientation about its axis. However, optionally, one or more of these torque functions may be defined according to a more arbitrary function, such as a polynomial, a sine function, a hyperbolic sine function, a tangent function, a hyperbolic tangent function, a log function, Hooke's Law, Coulomb's Law, Gauss's Law, etc. Alternatively, one or more of these functions may return a constant, indicating that no amount of torque will result in a displacement. Furthermore, one or more of these functions may be discontinuous or implement a logical algorithm with conditions, such as, “if (distance <0.0), then return MAX_REPULSIVE_FORCE; if (distance >=0.0 AND distance <0.05), then return SMALL_ATTRACTIVE_FORCE; if (distance >=0.05 AND distance <1.0), then return LARGE_ATTRACTIVE_FORCE; if (distance >=1.0), then return MAX_ATTRACTIVE_FORCE”.

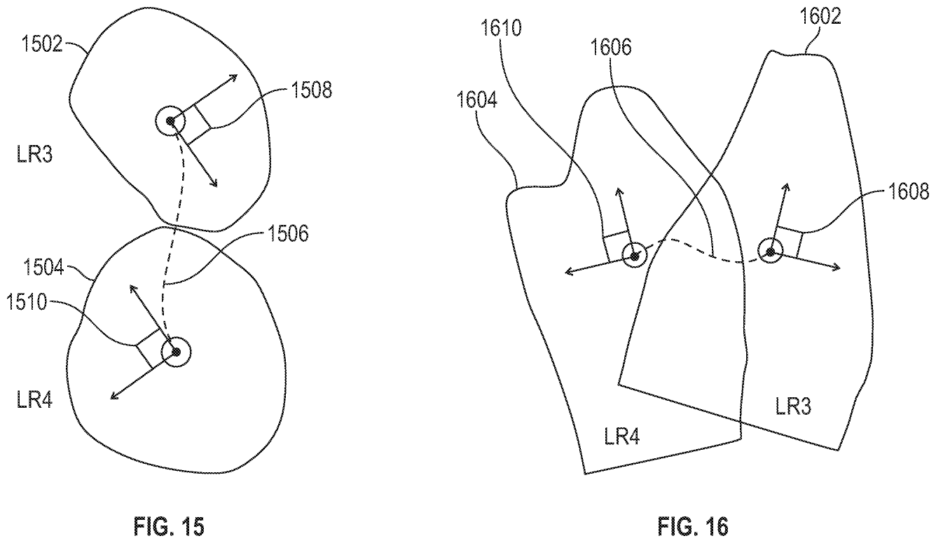

FIG. 15 shows an occlusal (top-down) view of 2 neighboring teeth, LR3 1502 and LR4 1504, in a maloccluded lower dental arch, a spring 1506 connecting teeth 1502 and 1504 at their centroids, and coordinate systems 1508 and 1510 at each of spring 1506's respective endpoints, defining the spring's orientation at these points. Spring 1506 in FIG. 15 is analogous to spring 1406 in FIG. 14, except spring 1406 is defined to be in a relaxed state as the teeth are in their target positions/orientations, while spring 1506 is in a stressed state as the teeth are displaced in their maloccluded positions. Spring 1506 is said to be deflected from its equilibrium form and therefore under stress, wherein the magnitude of the stress is defined by the function or model used to relate the force or moment to the linear or angular displacement, respectively, from equilibrium. In this example, spring 1506 is shown to be deformed by bending moments (as indicated by its curved shape) and by compression (as indicated by its shorter length compared with spring 1406). In some embodiments, spring 1506 may be modeled as a coil spring, a leaf spring, a rod, a bar, a wire, or a mathematical construct such as a spline or parametric curve, whereby its endpoints are in a fixed orientation while its midsection can vary continuously in its orientation between them. In other embodiments, spring 1506 represents a linear spring between 2 freely pivoting endpoints, while coordinate systems 1508 and 1510 represent degrees of deflection from equilibrium positions defined by the axis of spring 1506. For example, since the distal (axial) vector of coordinate system 1508 is somewhat rotated about its occluso-gingival (vertical) axis from a straight line connecting the centroids of teeth 1502 and 1504 (the origins of coordinate systems 1508 and 1510), tooth 1502 may be said to have a spherical or rotary spring deflected about its occluso-gingival axis. If there are also rotations about the mesio-distal and/or occluso-gingival vectors, then the rotations may be combined by virtue of Euler angles or quaternions into a single rotation about a different axis. Furthermore, the rotations may be combined with one or more translational components into a single 4×4 matrix or 3D Affine transformation (transform). In this example, because the coordinate systems are defined according to generally accepted rules of dental anatomy, where the origin is located near the tooth centroid, the mesial vector is oriented along a mesio-distal axis of the tooth, the labial vector is oriented along a facial axis of the tooth, and the occlusal vector is oriented along an occluso-gingival or longitudinal axis of the tooth, the intercentroid spring and optional spherical or rotary spring(s) are deflected in such a way that the teeth will have a tendency to move toward a more desirable arrangement, such as that shown in FIG. 14 by teeth 1402 and 1404. Spring 1506 has a tendency, upon iterative recomputation and movement, to relax into a straighter configuration, such as that shown in FIG. 14 by spring 1406. In general, force vs. displacement functions such as those defined by Hooke's Lay, Coulomb's Law, Gauss's Law, or the like, tend to move endpoints or particles closer together as potential energy in the system is reduced (assuming laws of attraction, not repulsion). In so acting, spring 1506 will tend to lengthen (due to its equilibrium form being longer) and straighten out, thereby bringing teeth 1502 and 1504 into alignment along their mesio-distal axes. Collision may be substantially, although not entirely, avoided by the lengthening of spring 1506 as it relaxes. Alternatively, spring 1506 may be treated as a linear spring with free ends, acting only along a straight axis line, and coordinate systems 1508 and 1510 treated as rotary springs that serve to rotate teeth 1502 and 1504 about their centroids respectively and independently. In effect, both of tooth 1502 and tooth 1504 will tend to rotate counterclockwise relative to the axis of spring 1506 until their respective distal and mesial axes align. Note, however, that since each tooth may have a plurality of springs associated with or attached to it, a system of forces and moments may be acting on the tooth to affect its motion.

In such cases, for example as shown in FIG. 14 where all but the distalmost teeth have 2 springs attached to their centroids, a vector sum of all forces and moments may be computed to determine a single, resultant force vector and moment vector. These forces and moments may be combined into a single 4×4 matrix, for example. Furthermore, these forces and moments may be further used to compute a separate 4×4 matrix or 3D Affine transformation (transform) indicating a movement of the tooth. The movement transform may be defined as a function or relation or model that determines a new tooth position based on the system of forces applied to it. During each iteration or stage or orthodontic treatment, wherein there could be many small iterations of computation and simulation between clinically significant stages where appliances are produced, a system of forces and moments on each tooth may be computed as vectors sums of one or more springs associated with the tooth, collisions may be determined and optionally contribute to these vector sums, resultant vectors or transforms may be computed from the system of forces and moments, and geometric movement vectors or transforms may be computed from the force- and moment-based resultant vectors or transforms. The teeth are subsequently moved according to the movement vectors or transforms, and the process is repeated until a certain limit is reached, such as a fixed number of iterations, a minimum displacement threshold, a minimum positional error, a minimum stress in the springs, diminished tooth movement per iteration, etc.

FIG. 16 shows a mesial (side or lateral) view of 2 neighboring teeth, LR3 1602 and LR4 1604, in a maloccluded lower dental arch, a spring 1606 connecting teeth 1602 and 1604 at their centroids, and coordinate systems 1608 and 1610 at each of spring 1606's respective endpoints, defining the spring's orientation at these points. Spring 1606 in FIG. 16 is analogous to spring 1505 in FIG. 15 and spring 1406 in FIG. 14, except spring 1406 is defined to be in a relaxed state as the teeth are in their target positions/orientations, while spring 1606 is in a stressed state as the teeth are displaced in their maloccluded positions. In this example, because teeth 1602 and 1604 and their respective coordinate systems 1608 and 1610 are rotated labially and lingually, respectively, of their target positions as defined in FIG. 14, spring 1606 is twisted or said to be torqued or in torsion from its relaxed state, which would otherwise put teeth 1602 and 1604 in substantially upright orientations. Furthermore, spring 1606 is deformed by bending moments due to teeth 1602 and 1604 being labially and lingually translated, respectively, from their target positions. Similarly, teeth 1602 and 1604 may be slightly occluso-gingivally (vertically) displaced from their target positions, which adds another dimension of deformation to spring 1606. As described in relation to FIG. 15, spring 1606, and optional rotary springs based on tooth coordinate systems 1608 and 1610, tend to relax and, in so doing, transform the teeth 1602 and 1604 from their current maloccluded positions shown in FIGS. 15 & 16 to their target positions shown in FIG. 14. As described earlier, their transformations are according to an iterative process, which may serve to substantially avoid collisions between the teeth.

As in other examples described herein, the length of spring between tooth centroids may be adjusted for different scenarios. For example, the relaxed length of the spring may be greater than the sum of the neighboring partial tooth widths so that a gap is left between the teeth, or the relaxed length is equal to the sum of the neighboring partial tooth widths so that the teeth contact precisely when the spring is at rest, or the relaxed length is less than the sum of the neighboring partial tooth widths so that the spring is in tension when the teeth are in contact. The spring length may be adjusted to control effects related to static friction or texture or bumps on the teeth, so as to overcome blocking forces that prevent teeth from sliding into their desired final positions after making contact.

In some examples, it may be desirable to combine these types of springs with other types of springs defined above into a system of springs to implement complex functions designed to respond to various interferences as teeth move in and out of contact. For example, a set of linear springs with unconstrainted endpoints connected to labial and lingual attachment points may be used in addition to the centroid-connected springs with fully constrained endpoints (constrained by a pair of vectors constituting a coordinate system and resembling a spherical spring or a system of 3 rotary springs). In so doing, the net force or moment function acting on any given tooth may be dynamic, meaning that it can change depending on the relative positions of the teeth and how they might interact in contact with one another, not just as a function of the distances between the teeth or the orientations of the teeth or the orientations of the endpoints. In such a system, the net forces and moments on any given tooth could be a function of the state of a plurality of springs, the positions and orientations of neighboring teeth, and the shapes of the dental surfaces that contact or don't contact one another.

1. A method of generating one or more transformations for an orthodontic treatment, the method comprising:
receiving, by processing circuitry of a computing device, a digital representation of a patient's dentition, wherein the representation comprises one or more teeth;
applying, by the processing circuitry, a force directed graph (FDG) to the one or more teeth;
computing, by the processing circuitry, one or more representations of physical interactions on at least one tooth, wherein each representation comprises a force, a moment, or both;
applying, by the processing circuitry, the one or more representations of physical interactions to the at least one tooth;
generating, by the processing circuitry, a transform for the at least one tooth that describes an application of the one or more physical interactions to the at least one tooth; and
applying, by the processing circuitry, the transform to the at least one tooth.

2. The method of claim 1, wherein the applying comprises computing at least one of a translation or a rotation of the tooth based, at least in part, on the one or more representations of physical interactions.

3. The method of claim 2 wherein each respective tooth of the one or more teeth is represented by a corresponding node in the FDG.

4. The method of claim 3, further comprising modeling, by the processing circuitry, at least one respective force acting on a respective node in the FDG based on at least one of Gauss's Law, a Universal Law of Gravitation, Hooke's Law, a nuclear force, or a model that describes at least one of a force or a moment as it relates to a displacement.

5. The method of claim 1, wherein the transform for at least one tooth comprises a setup transformation for orthodontic aligner treatment.

6. The method of claim 5, wherein the setup transformation comprises transforming the tooth into a target arrangement of the teeth which includes a description of the pose of the tooth after completion of at least a portion of an orthodontic treatment of the patient.

7. The method of claim 5, wherein the setup transformation comprises transforming the tooth into a target arrangement of the teeth which includes a description of the pose of the tooth during a course of an orthodontic treatment of a patient.

8. The method of claim 1, wherein the digital representation includes one or more of: (i) one or more vectors P containing at least one value pertaining to at least one method of computing a tooth dimension, (ii) one or more vectors Q containing at least one value pertaining to at least one method of computing a distance between adjacent teeth, (iii) one or more 3D representations of one or more teeth, (iv) one or more vectors K containing at least one data value from a set of procedure parameters, (v) one or more vectors L containing at least one data value from a set of doctor preferences, (vi) one or more vectors M containing at least one flag indicating whether at least one tooth is at least one of fixed, pinned, pontic, extracted, implanted, or missing, (vii) one or more vectors N containing at least one value pertaining to the position of at least one tooth, (viii) one or more vectors O containing at least one value pertaining to the orientation of at least one tooth, (ix) one or more vectors R containing at least one of tooth name, designation, tooth type, or tooth classification, (x) one or more vectors S containing at least one value pertaining to at least one metric pertaining to at least one tooth, or (xi) one or more vectors U containing at least one value pertaining to at least one of a mesial and a distal IPR value for at least one tooth.

9. The method of claim 1, wherein the one or more representations of physical intersections are defined as acting between teeth in a substantially similar stage of treatment.

10. The method of claim 1, wherein the one or more representations of physical intersections are defined as acting between the same teeth in different stages of treatment.

11. The method of claim 1, wherein one or more of the physical interactions act on a first endpoint associated with a first tooth and a second endpoint associated with a second tooth, and wherein the second tooth is in substantially the same stage of treatment as the first tooth.

12. The method of claim 1, wherein one or more of the physical interactions act on a first endpoint associated with a first tooth and a second endpoint associated with a second tooth, and wherein the second tooth is in a different stage of treatment as the first tooth.

13. The method of 12, wherein the first tooth and the second tooth have the same identity.

14. The method of claim 1, wherein the one or more physical intersections includes a collision involving a tooth and one or more other 3D representations of oral care data in the dentition.

15. The method of claim 14, wherein generating the transform for the at least one tooth is further based at least in part on one or more collisions between the at least one tooth and one or more other 3D representations of oral care data in the dentition.

16. The method of claim 14 wherein the one or more representations of oral care data include at least one of a tooth, a pontic, a block of teeth, a crown, a root, a bridge, an implant, a bite block, an attachment, a bracket, an appliance, a restoration, or at least a portion of cortical bone.

17. A method of claim 9, wherein at least one of the first endpoint or the second endpoint is proximal to at least one of a surface of a tooth crown, a surface of a tooth root, a crown centroid, a root centroid, a root center of resistance, a root center of moments, a root center of area, a tooth contact point, an interproximal surface, at least a portion of an appliance, or at least one 3D representation of oral care data.

18. A method of claim 10, wherein at least one of the first endpoint or the second endpoint is proximal to at least one of a surface of a tooth crown, a surface of a tooth root, a crown centroid, a root centroid, a root center of resistance, a root center of moments, a root center of area, a tooth contact point, an interproximal surface, at least a portion of an appliance, or at least one 3D representation of oral care data.

19. The method of claim 1, wherein the computing device is deployed in a clinical context, and wherein the method is performed in near real-time during an encounter with a patient.

20. A computing device for generating a transformation for oral care treatment, the computing device comprising:
interface hardware configured to receive an orthodontic treatment representation;
processing circuitry configured to:
receive a digital representation of a patient's dentition, wherein the representation comprises one or more teeth;
apply a force directed graph (FDG) to the one or more teeth;
compute one or more representations of physical interactions on at least one tooth, wherein each representation comprises a force, a moment, or both;
apply the one or more representations of physical interactions to the at least one tooth;
generate a transform for the at least one tooth that describes an application of the one or more physical interactions to the at least one tooth;
apply the transform to the at least one tooth; and
a memory unit configured to store the orthodontic treatment representation.
